(19) United States
(12) Patent Application Publication
(10) Pub. No.: US 2024/0423954 A1
(43) Pub. Date: Dec. 26, 2024

(54) Chlorthalidone Compositions And Methods

(71) Applicant: Nivagen Pharmaceuticals, Inc., Sacramento, CA (US)

(72) Inventors: Govind R. Jagadale, Sacramento, CA (US); Dasaradhi Lakkaraju, Sacramento, CA (US); Bala Tripura Sundari Chodavarapu, Davis, CA (US); Niravkumar Prajapati, Sacramento, CA (US); Anand Shukla, Denver, CO (US); Jay Shukla, Sacramento, CA (US); Robert Miller, Sacramento, CA (US); Niravbhai Jayantibhai Patel, Sacramento, CA (US); Maheshkumar Kalubhai Bhalaria, Sacramento, CA (US)

(73) Assignee: Nivagen Pharmaceuticals, Inc., Sacramento, CA (US)

(21) Appl. No.: 18/594,178

(22) Filed: Mar. 4, 2024

Related U.S. Application Data
(63) Continuation-in-part of application No. 17/331,011, filed on May 26, 2021, now Pat. No. 11,957,658.
(60) Provisional application No. 63/032,434, filed on May 29, 2020.

(51) Int. Cl. A61K 31/4035; A61K 9/00; A61K 47/02; A61K 47/10; A61K 47/12; A61K 47/14; A61K 47/24; A61K 47/34; A61K 47/38

(52) U.S. Cl. CPC A61K 31/4035; A61K 9/0053; A61K 47/02; A61K 47/10; A61K 47/12; A61K 47/14; A61K 47/24; A61K 47/34; A61K 47/38

(57) ABSTRACT

Compositions of a stabilized chlorthalidone suspension and methods for making a stabilized chlorthalidone suspension include chlorthalidone along with a solubilizing and/or wetting agent, a suspending agent, and a viscosity increasing agent and/or an anti-caking agent, wherein the stabilized chlorthalidone suspension is a uniform dispersion with consistent concentration of chlorthalidone throughout the composition and storage.

### CROSS-REFERENCE DATA

This application is a continuation-in-part application of allowed U.S. patent application with the Ser. No. 17/331,011, filed 26 May 2021, which claims priority to U.S. Provisional Patent Application with the Ser. No. 63/032,434, filed 29 May 2020, each of which is incorporated by reference herein.

### FIELD OF THE INVENTION

The field of the invention is liquid suspensions of chlorthalidone, especially as they relate to stable suspensions of uniformly dispersed chlorthalidone.

### BACKGROUND

The following description includes information that may be useful in understanding the present invention. It is not an admission that any of the information provided herein is prior art or relevant to the presently claimed invention, or that any publication specifically or implicitly referenced is prior art.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Chlorthalidone is a prescription diuretic drug used to treat high blood pressure (hypertension). Chlorthalidone is also used to reduce extra salt and water in the body caused by conditions such as heart failure, liver disease, and kidney disease, and is also used to treat diabetes. Currently, chlorthalidone is available as a prescription drug, and it is only manufactured as an oral tablet of 25 mg or 50 mg doses (Chlorthalidone Tablets USP, 25 mg and 50 mg).

Effective use and administration of a 25 mg or 50 mg Chlorthalidone tablet is prone to errors including miscalculated or imprecise doses. For example, an oral tablet formulation may need to be cut in half for administration of a prescribed dose amount; however accurately splitting the tablet can be problematic for some dosage amounts. Furthermore, while dosages which are multiples of 25 or 50 can be administered in oral tablet form, lower dosages (e.g., less than 25 mg) or dosages between 25 and 50 mg or higher than 50 mg and especially those which are not multiples of 25, are not easy to obtain from the 25 mg or 50 mg tablets.

Thus, there is still a need for an oral formulation of chlorthalidone for more precise and effective oral administration of the chlorthalidone at various dosage levels.

### SUMMARY OF THE INVENTION

The inventive subject matter provides compositions and methods of a liquid suspension formulation of chlorthalidone. In particular, the contemplated composition is a stabilized chlorthalidone suspension including chlorthalidone at a concentration of between 1 to 20 mg/mL, a solubilizing agent and/or wetting agent, at least one suspending agent, at least one viscosity increasing agent or at least one anti-caking agent, and water.

Notably, despite the propensity of ingredients in suspension to settle, the dispersion of the chlorthalidone API presented herein remained substantially uniform throughout over extended periods, thus allowing for accurate dispensing and dosing. For example, a first sample concentration of the chlorthalidone in a first sample volume of the composition taken before storage differs from a second sample concentration in a second volume of the composition after storing for at least 3 hours, or even at least 6-12 hours by no more than 5%. More preferably, the first sample concentration of the chlorthalidone in the first sample volume of the composition taken before storage differs from the second sample concentration in the second sample volume of the composition after storage by no more than 2.5%. Most preferably, the first sample concentration of the chlorthalidone in the first sample volume of the composition taken after stirring to reach uniform dispersion and before storage differs from the second sample concentration in the second sample volume of the composition taken after storage by no more than 1.0%. Typically, the first sample volume is obtained from a section of the stabilized chlorthalidone suspension that is above or below and does not overlap in a vertical direction with the part of the stabilized chlorthalidone suspension from which the second sample volume is obtained.

In some embodiments, the stabilized chlorthalidone suspension includes chlorthalidone, a solubilizing or wetting agent, at least one suspending agent, at least one viscosity increasing agent or at least one anti-caking agent, water, and further includes an anti-foaming agent, at least one antimicrobial, and at least one sweetening agent.

Preferably, the chlorthalidone is present in the stabilized suspension at a concentration of 1 to 20 mg/mL. More preferably, the chlorthalidone is present in the stabilized suspension at or between about 5 to 15 mg/mL. Most preferably, the chlorthalidone is present in the stabilized suspension at about 10 mg/mL.

The solubilizing or wetting agent in the stabilized suspension may be any suitable poloxamer, which is a wetting (e.g., emulsifying) agent. Preferably, the wetting agent is poloxamer 188, poloxamer 124, or poloxamer 237. Typically, the wetting agent is poloxamer 188. More typically, the wetting agent is poloxamer 188 at a concentration at or between 5 to 20 mg/mL. Most typically, the wetting agent is poloxamer 188 at a concentration of about 10 mg/mL.

The suspending agent in the stabilized suspension may be a polysaccharide suspending agent and/or a synthetic suspending agent. In preferred embodiments, the suspending agent is one or more polysaccharide suspending agent selected from microcrystalline cellulose (MCC), acacia gum, tragacanth gum, xanthan gums, starch, or alginates. More preferably, the polysaccharide suspending agent is MCC. Typically, the MCC is present in the stabilized suspension at a concentration of between about 10 to 20 mg/mL. More typically, the MCC is present in the stabilized suspension at a concentration of 20 mg/mL.

The viscosity increasing agent and/or anti-caking agent in the stabilized suspension may be selected from hydroxyethylcellulose (HEC), silicon dioxide, and/or polyethylene glycol (PEG). Notably, a suitable viscosity increasing agent may also be an anti-caking agent. Typically, the viscosity agent or anti-caking agent is present in the stabilized suspension at or between 0.1% to 5% by weight (wt %). Preferably, viscosity increasing agent and/or anti-caking agent in the stabilized suspension includes HEC and silicon dioxide. In preferred embodiments, the HEC (for HEC having a viscosity of 400 centipoise (cps)) is present in the stabilized suspension at a concentration at or between about 1 to 30 mg/mL or at or between 1 to 5 mg/mL, and the silicon dioxide (alone or together with the HEC) is present in the stabilized suspension at a concentration at or between about 10 to 45 mg/mL or 15 to 25 mg/mL. In more preferred embodiments, in addition to HEC and silicon dioxide, polyethyleneglycol (PEG) is included in the stabilized suspension at or between about 25 and 50 mg/mL. The PEG may have a molecular weight at or between 300 to 1,000 gram/mol (g/mol). For HEC having a viscosity of 2,000 cps, an amount from about 0.25 to 1.25 mg/mL HEC is added to the stabilized suspension. More preferably, the amount of HEC (at 2,000 cps) is about 0.25 mg/mL. In some embodiments, the stabilized chlorthalidone suspension also includes a flavor, a flavoring agent, a coloring agent, and/or a dye.

The inventive subject matter includes methods for making the contemplated stabilized chlorthalidone suspension. A contemplated method includes preparing the suspension using water. Typically, the method includes adding to water: a solubilizing and/or a wetting agent with stirring, adding a suspending agent with stirring and homogenizing, adding at least one viscosity increasing agent and/or at least one caking agent with stirring and homogenizing, and adding a solubilized solution of chlorthalidone with stirring and homogenizing.

In preferred embodiments, a contemplated method for making the stabilized chlorthalidone suspension includes making a first mixture by adding a solubilizing and/or a wetting agent to a first volume of water having a temperature of between about 25° C. to 30° C. with stirring, adding a suspending agent to the first volume of water with stirring and homogenizing, and adding at least one viscosity increasing agent or at least one anti-caking agent to the first volume of water with stirring and homogenizing. The contemplated method also includes making a second mixture by adding a sweetening agent to a second volume of water with stirring, adding a viscosity increasing agent to the second volume of water with stirring, and adding chlorthalidone at between about 5 to 20 mg/mL to the second volume of water with stirring and homogenizing. The stabilized chlorthalidone suspension is formed by combining the first mixture and the second mixture with stirring to form a third mixture and homogenizing. In some embodiments, additional water may be added to bring the mixture to the calculated volume for the desired concentration of components including chlorthalidone.

In typical embodiments of any of the methods disclosed above or any method set forth below, the solubilizing and/or a wetting agent may be one of poloxamer 188, poloxamer 124, or poloxamer 237. In more typical embodiments, the solubilizing and/or a wetting agent is poloxamer 188. Preferably, the poloxamer 188 is added to the water or the first volume of water to have a final concentration of between about 5 mg/mL to about 20 mg/mL. Most preferably, the final concentration of the poloxamer 188 is 10 mg/mL.

In further typical embodiments of the method disclosed above or any method set forth below, the suspending agent is a polysaccharide suspending agent and/or a synthetic suspending agent. Examples of a polysaccharide suspending agent include microcrystalline cellulose (MCC), acacia gum, tragacanth gum, xanthan gums, starch, or alginates. Preferably, the suspending agent is MCC added to the water or the first volume of water to have a final concentration of between about 10 to 20 mg/mL. Most preferably, the final concentration of the MCC added to the water or the first volume of water is about 20 mg/mL.

In still further typical embodiments of the method disclosed above or any method set forth below, the at least one viscosity increasing agent or the at least one anti-caking agent is hydroxyethylcellulose and/or silicon dioxide. Preferably, the at least one viscosity increasing agent or the at least one anti-caking agent is added to the water or the first volume of water to have a final concentration of between about 0.1 to 5% by weight (wt. %).

In more preferred embodiments, the contemplated method for making the stabilized chlorthalidone suspension includes making the first mixture as disclosed above, wherein making the first mixture further includes heating the water to about 80° C. to 85° C., adding at least one antimicrobial preservative to the heated water with stirring, and ambiently cooling the water to about 25° C. to 30° C., all of which occur prior to adding the solubilizing and/or a wetting agent. Preferably, the at least one antimicrobial is methyl paraben, propyl paraben, and/or potassium sorbate.

In additional preferred embodiments, making the first mixture further includes adding an anti-foaming agent to the first volume of water with stirring. Preferably the anti-foaming agent is simethicone. In other preferred embodiments, making the first mixture further includes adding a sweetening agent with stirring and homogenizing. Typically, the sweetening agent is sucrose, sucralose, glycerin, stevia, and/or sorbitol.

In still other embodiments, the contemplated stabilized chlorthalidone suspension includes chlorthalidone at a concentration of between 1 to 20 mg/mL, a solubilizing and/or a wetting agent, at least one suspending agent, at least two viscosity increasing agents, water, an anti-foaming agent, at least one antimicrobial, a buffering agent, and at least one sweetening agent, wherein a first concentration of the chlorthalidone in a first volume of the composition differs from a second concentration in a second volume of the composition by 1% or less.

With reference to the above stabilized chlorthalidone suspension, in preferred embodiments, the chlorthalidone is at a concentration of 5 mg/mL or 10 mg/mL, the solubilizing and/or a wetting agent is propylene glycol (PPG), the at least one suspending agent is microcrystalline cellulose (MCC), the at least two viscosity increasing agents are selected from hydroxyethylcellulose (HEC), xanthan gum, and glycerin, the water is deionized water, the anti-foaming agent is simethicone, the buffering agent is citric acid anhydrous, and the sweetening agent is sucralose.

In related embodiments, a method for making the above stabilized chlorthalidone suspension includes forming a first mixture including heating a first volume of water and a first solubilizing agent to 80° C. to 85° C., wherein the first volume of water is 40% to 60% of the volume of the stabilized chlorthalidone suspension and the solubilizing agent is 0.5%. The first mixture also includes adding at least one anti-microbial/preservative to the first mixture with stirring at 80° C. to 85° C., cooling the first mixture without applying an external source to 25° C. to 30° C., adding an anti-foaming agent to the first mixture with homogenization, wherein the anti-foaming agent is added at 50 to 75% of the concentration of the anti-foaming agent in the stabilized chlorthalidone suspension. The first mixture also includes adding a suspending agent and a sweetening agent to the first mixture either sequentially or simultaneously with stirring and/or homogenization, adding an anti-caking agent to the first mixture with stirring and/or homogenization, adding a first viscosity increasing agent to the first mixture with stirring, and adding a second viscosity increasing agent to the first mixture with stirring.

The method for making the above stabilized chlorthalidone suspension also includes forming a second mixture including adding 50 to 25% of the concentration of the anti-foaming agent in the stabilized chlorthalidone suspension to a second volume of water with stirring, wherein the second volume of water is 40 to 60% of the volume of the stabilized chlorthalidone suspension, adding a second solubilizing and/or a second wetting agent to the second mixture with stirring, and adding chlorthalidone to the second mixture with stirring.

The method for making the above stabilized chlorthalidone suspension also includes forming a third mixture including mixing the first mixture and the second mixture with stirring and adding a buffering agent and optionally a flavor to the third mixture with stirring.

With reference to the above method for making the stabilized chlorthalidone suspension, in preferred embodiments, the method includes the first solubilizing and/or a wetting agent is polypropylene glycol (PPG), the at least one anti-microbial/preservative is methyl paraben, poly paraben, and/or potassium sorbate, the anti-foaming agent is simethicone, the suspending agent is microcrystalline cellulose (MCC), the anti-caking agent is hydroxyethylcellulose (HEC), the first viscosity increasing agent is xanthan gum, the second viscosity increasing agent is glycerin, the second solubilizing and/or a wetting agent is poloxamer 188, and the buffering agents.

In still other preferred embodiments, any of the above methods for making a stabilized chlorthalidone suspension include adding a flavor, a flavoring agent, a coloring agent, and/or a dye the water, or one of the first mixture, the second mixture, or the third mixture with stirring.

Preferably, the contemplated methods of making a stabilized chlorthalidone suspension render a suspension wherein a first concentration of the chlorthalidone in a first volume of the stabilized chlorthalidone differs from a second concentration in a second volume of the composition by 2.5% or less. More preferably, the first concentration of the chlorthalidone in a first volume of the stabilized chlorthalidone differs from a second concentration in a second volume of the composition by 1.0% or less.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A is a flow chart depicting exemplary method steps for preparing a first mixture (Mixture 1) of Process A, according to an embodiment of the present invention.

FIG. 1B is a flow chart depicting exemplary method steps for preparing a second mixture (Mixture 2) of Process A, according to embodiments of the present invention.

FIG. 1C is a flow chart depicting exemplary method steps of Process A, for mixing the first mixture of FIG. 1A and the second mixture of FIG. 1B, according to embodiments of the present invention.

FIG. 1D is a flow chart depicting exemplary method steps for preparing a first mixture (Mixture 1) of Process B, according to embodiments of the present invention.

FIG. 1E is a flow chart depicting exemplary method steps for preparing a second mixture (Mixture 2) of Process B, according to embodiments of the present invention.

FIG. 1F is a flow chart depicting exemplary method steps of Process B, for mixing the first mixture of FIG. 1D and the second mixture of FIG. 1E, according to embodiments of the present invention.

FIG. 2 is a graph of exemplary chlorthalidone suspensions as indicated, as disclosed herein and prepared according to embodiments of the present invention.

FIGS. 3A-3K are tables showing exemplary variations in the formulations of the dispersed chlorthalidone suspension, according to embodiments of the present invention.

FIGS. 4A-4F are tables showing exemplary stability data for batch NCH1972 at 25° C. (FIGS. 4A-4C) and 40° C. (FIGS. 4D-4F), according to embodiments of the present invention.

FIGS. 5A-5F are tables showing exemplary stability data for batch NCH1979 at 25° C. (FIGS. 5A-5C) and 40° C. (FIGS. 5D-5F), according to embodiments of the present invention.

FIGS. 6A-6F are tables showing exemplary stability data for batch NCH1984 at 25° C. (FIGS. 6A-6C) and 40° C. (FIGS. 6D-6F), according to embodiments of the present invention.

FIGS. 7A-7F are tables showing exemplary stability data for batch NCH19122 at 25° C. (FIGS. 7A-7C) and 40° C. (FIGS. 7D-7F), according to embodiments of the present invention.

FIG. 8 is a graph depicting comparative dissolution profiles of milled and unmilled chlorthalidone.

FIGS. 9A-9D are tables showing exemplary stability data for milled API (9A) and unmilled API (9C) at 40° C., and milled API (9B) and unmilled API (9D) at 25° C., according to embodiments of the present invention.

### DETAILED DESCRIPTION

The inventors have discovered compositions and methods for a chlorthalidone suspension for oral administration. In particular, the suspensions according to the inventive subject matter provide an unexpected stable dispersion of chlorthalidone in a liquid formulation. For example, the stably dispersed chlorthalidone suspension is characterized by having a remarkably low settling rate, rendering a liquid formulation having the chlorthalidone active ingredient uniformly dispersed at the same concentration or approximately the same concentration throughout the suspension such that any sample of the suspension taken corresponds to the same or approximately the same concentration of another sample taken, even after extended periods of time. Accordingly, one should appreciate that the disclosed stabilized suspensions of chlorthalidone provide a chlorthalidone formulation that is easily and accurately dosed during manufacture as well as use with a patient.

In particular, the contemplated composition is a stabilized chlorthalidone suspension including chlorthalidone as the active pharmaceutical ingredient (API) at a concentration of between 1 to 20 mg/mL, with excipients including at least a solubilizing and/or a wetting agent, at least one suspending agent, at least one viscosity increasing agent or at least one anti-caking agent, and water. Preferably, the presently disclosed composition and methods provide for a chlorthalidone suspension having a first concentration of the chlorthalidone in a first volume of the composition that differs from a second concentration in a second volume of the composition by no more than 5%. In other words, the chlorthalidone suspension is at least 95% homogenous with respect to the concentration of chlorthalidone found throughout the dispersion. Preferably, the chlorthalidone suspension is at least 95% homogenous with respect to all components. As disclosed herein, the first concentration of the suspension may differ from the second concentration by no more than 0.5%. Accordingly, the contemplated chlorthalidone suspension may have a first concentration of chlorthalidone in a first volume of the composition that differs from a second concentration in a second volume of the composition by no more than 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.5%, 3.6%, 3.7%, 3.8%, 3.9%, 4.0%, 4.1%, 4.2%, 4.3% 4.4% 4.5% 4.6%, 4.7%, 4.7% 4.9% or 5.0%.

In some embodiments, it is contemplated that the suspension maintains uniform concentration over extended storage times. For example, the chlorthalidone formulations presented herein may be stored over a time period of at least 20 min, at least 30 min, at least 40 min, at least 50 min, at least 1 hr, at least 2 hrs, at least 3 hrs, at least 4 hrs, at least 5 hrs, at least 6 hrs, at least 9 hrs, at least 12 hrs, at least 18 hrs, at least 24 hrs, at least 36 hrs, at least 48 hrs, at least 72 hrs, at least 4 days, at least 5 days, at least 6 days, at least 7 days, at least 10 days, at least 20 days, at least 30 days, at least 45 days, at least 2 months, at least 3 months, at least 4 months, at least 5 months, or at least 6 months. During such time period, it is contemplated that after initial stirring to generate a homogenous suspension, the chlorthalidone in a first sample taken from the composition immediately after stirring will differ from a second sample in a second volume of the composition by no more than 10%, or no more than 9%, or no more than 8%, or no more than 7%, or no more than 6%, or no more than 5%, or no more than 4%, or no more than 3%, or even less. Advantageously, such stable suspensions will not only ensure uniform drug concentration during dispensing from a large holding tank to individual use containers in a production environment, but also help maintain uniform drug concentration at the point of use to so ensure proper individual dosing of the drug. In further examples, a second sample of suspension for detecting of the chlorthalidone concentration is contemplated to be taken after a period of composition storage time of at least 10 min after taking the first sample from which the chlorthalidone concentration is determined.

Preferably, the chlorthalidone is present in the stabilized suspension at a concentration of 1 to 20 mg/mL. More preferably, the chlorthalidone is present in the stabilized suspension at or between about 5 to 15 mg/mL. Most preferably, the chlorthalidone is present in the stabilized suspension at about 5 mg/mL or 10 mg/mL.

Notably, initial experiments revealed an expected outcome, in that the chlorthalidone API was not stable in solution as it would agglomerate and cake. Accordingly, for stabilizing the chlorthalidone in a stabilized suspension, the inventors contemplated combining the chlorthalidone with select excipients. These excipients include at least a solubilizing and/or a wetting agent, at least one suspending agent, at least one viscosity increasing agent or at least one anti-caking agent, and water. The suspension may also further include an anti-foaming agent, at least one antimicrobial, and at least one sweetening agent.

Unless disclosed otherwise, the contemplated suspension may include any excipient disclosed herein at a final concentration of no more than its inactive ingredient (IIG) limit as set by the U.S. Food and Drug Administration (FDA) for that excipient compound. For example, as set forth in Tables 1A and 1B below, exemplary excipients are listed along with the corresponding IIG limit, function, and concentration (e.g., final concentration) in each of the 10 mg/mL chlorthalidone suspension including Formulation 1 (NCH1972), Formulation 2 (NCH1979), and Formulation 3 (NCH1984) of Process A and the excipients added to a 5 mg/mL chlorthalidone suspension prepared according to Process B, as disclosed herein below.

The solubilizing and/or a wetting agent in the stabilized chlorthalidone suspension may be any suitable poloxamer (a wetting agent). For example, the wetting agent may be poloxamer 188, poloxamer 124, or poloxamer 237. As used herein, the wetting agent is poloxamer 188. Poloxamer 188 may be used at a final concentration at or between about 5 to 20 mg/mL. Preferably, poloxamer 188 is present in the stabilized suspension at a concentration of about 10 mg/mL.

The suspending agent in the stabilized chlorthalidone suspension may be any suitable suspending agent. In general, suspending agents include polysaccharide suspending agents, synthetic suspending agent, and salts. However, salt suspending agents are not suitable for use with chlorthalidone as salt is known to increase hypertension and therefore would be counterproductive for the intended treatment. Accordingly, suspending agents for the contemplated chlorthalidone suspension include polysaccharide suspending agents and synthetic suspending agents. Preferably, the polysaccharide or synthetic suspending agents are not charged at the pH of the chlorthalidone suspension which may have a pH of between 3.0 and 7.0. Typically, the pH of the chlorthalidone suspension is of between 4.0 and 6.0, and more typically the pH of the chlorthalidone suspension is of between 4.5 and 5.5. For example, polysaccharide suspending agents include one or more of microcrystalline cellulose (MCC), acacia gum, tragacanth gum, xanthan gums, starch, and/or alginates. For example, a polysaccharide suspending agent may be MCC and xanthan gum. In another example, the polysaccharide suspending agent is MCC. Typically, the MCC or MCC and xanthan gum are present in the stabilized suspension at a total concentration of between about 10 to 20 mg/mL. More typically, the MCC or MCC and xanthan gum are present in the stabilized suspension at a total concentration of about 20 mg/mL.

The viscosity increasing agent and/or anti-caking agent includes any suitable compound that increases viscosity and/or decreases caking. Notably, a suitable viscosity increasing agent may also be an anti-caking agent. The viscosity increasing agent and/or anti-caking agent in the stabilized suspension may be hydroxyethylcellulose (HEC), silicon dioxide, and/or polyethylene glycol (PEG). Typically, the viscosity agent or anti-caking agent is present in the stabilized suspension at or between 0.1% to 5% by weight (wt %). Preferably, viscosity increasing agent and/or anti-caking agent in the stabilized suspension includes HEC and/or silicon dioxide. In preferred embodiments, the HEC is present in the stabilized suspension at a concentration at or between about 0.25 to 1.25 mg/mL for HEC with a viscosity of 2,000 centipoise (cps) or at about 1 to 30 mg/mL for HEC with a viscosity of 400 cps. The silicon dioxide (alone or together with the HEC) is present in the stabilized suspension at a concentration at or between about 10 to 70 mg/mL, about 10 to 45 mg/mL, or about 15 to 25 mg/mL. In more preferred embodiments, in addition to HEC and silicon dioxide, polyethylene glycol (PEG) is included in the stabilized suspension at or between about 25 and 50 mg/mL. The PEG may have a molecular weight at or between 300 to 1,000 gram/mol (g/mol).

To enable more thorough mixing during production, the chlorthalidone suspension may also include an anti-foaming agent. Any suitable anti-foaming agent may used. Suitable anti-foaming agents include silicon-based (e.g., simethicone (simethicone), silicone, or polydimethylsiloxane), food grade mineral oils (e.g., mono- and diglycerides), or alginates (e.g., alginic acid). Preferably, the anti-foaming agent is simethicone (also referred to as simethicone). More preferably, simethicone is present in the stabilized suspension at a concentration of between about 5 mg/mL to about 9 mg/mL. Most preferably, simethicone is present at a concentration between about 8 mg/mL to about 9 mg/mL.

To prevent contamination and microbial growth in the stabilized chlorthalidone suspension, any suitable antimicrobial agent may be provided. Suitable antimicrobial compounds include one or more of methyl paraben, propyl paraben, and potassium sorbate (K-sorbate). Preferably, both methyl paraben and propyl paraben and/or potassium sorbate are present in the suspension. For example, methyl paraben may be present in the stabilized suspension at a concentration of between about 1 to 200 mg/mL, and propyl paraben may be present in the stabilized suspension at a concentration of between about 0.1 up to 40 mg/mL. Preferably, if both methyl paraben and propyl paraben are present in the suspension, an exemplary concentration of methyl paraben is about 1 mg/mL and an exemplary concentration of propyl paraben is about 0.2 mg/mL. Exemplary concentrations of potassium sorbate are of between about 1 to 5 mg/mL.

Additionally, sweetening agents may be added to the stabilized chlorthalidone suspension to mask unpleasant tastes or odors and facilitate consumption of the suspension by the subject (e.g., person or animal) in need of chlorthalidone. Accordingly, any suitable sweetening agent may be added to the stabilized suspension. Exemplary sweetening agents include sorbitol, sucrose, sucralose, stevia, and/or glycerin. Preferably, the concentration of sorbitol present in the stabilized suspension is of between about 50 mg/mL up to 975 mg/mL of a 70% sorbitol solution. More preferably, the concentration of sorbitol present in the stabilized suspension is about 200 mg/mL up to about 500 mg/mL, or about 300 mg/mL. Preferably, the concentration of sucrose present in the stabilizes suspension is of between about 50 mg/mL up to 500 mg/mL. More preferably, the concentration of sucrose is present in the stabilized suspension is about 100 mg/mL up to about 300 mg/mL, or about 150 mg/mL. Typically, sorbitol and sucrose are both present in the stabilized suspension. In other preferred embodiments, for diabetic patients in need of chlorthalidone, sucralose or stevia may be present with glycerin in the stabilized suspension.

While the color and flavor of a liquid formulation for oral administration does not affect the stability of an API and excipients in a suspension, both color and flavor may further help the subject in need of chlorthalidone consume the suspension more readily and/or with less stress. Accordingly, any food grade flavor or color agent may be included in the stabilized chlorthalidone suspension. Non-limiting examples of flavors include fruit flavors (e.g., strawberry, orange, grape, or cherry). Non-limiting examples of colors or dyes, include yellow, red, and blue, and all reasonable combinations thereof.

Advantageously, the inventors have contemplated a method for producing a stabilized chlorthalidone suspension having a thoroughly dispersed concentration of chlorthalidone such that any two sample volumes of the suspension have a concentration of chlorthalidone that differs from the other by no more than about 5%, and preferably no more than about 1%. Typically, a first sample volume is obtained from a section of the stabilized chlorthalidone suspension that is above or below and does not overlap in a vertical direction with the section of the stabilized chlorthalidone suspension from which a second sample volume is obtained. For example, the first sample volume may be obtained from the bottom section of the stabilized chlorthalidone suspension with the second sample volume being obtained from the top section of the stabilized chlorthalidone suspension, and the concentration of chlorthalidone in each of the two sample volumes does not differ by more than 5%, thereby indicating that the chlorthalidone is homogenously dispersed in the suspension and does not readily settle after an initial mixing (e.g., shaking). Such a thorough dispersion has minimal or no lumps or caking. Accordingly, the contemplated method for making the stabilized chlorthalidone suspension includes preparing the suspension using water and adding to the water: a solubilizing and/or a wetting agent with stirring, adding a suspending agent with stirring and homogenizing, adding at least one viscosity increasing agent and/or at least one caking agent with stirring and homogenizing, and adding a solubilized solution of chlorthalidone with stirring and homogenizing.

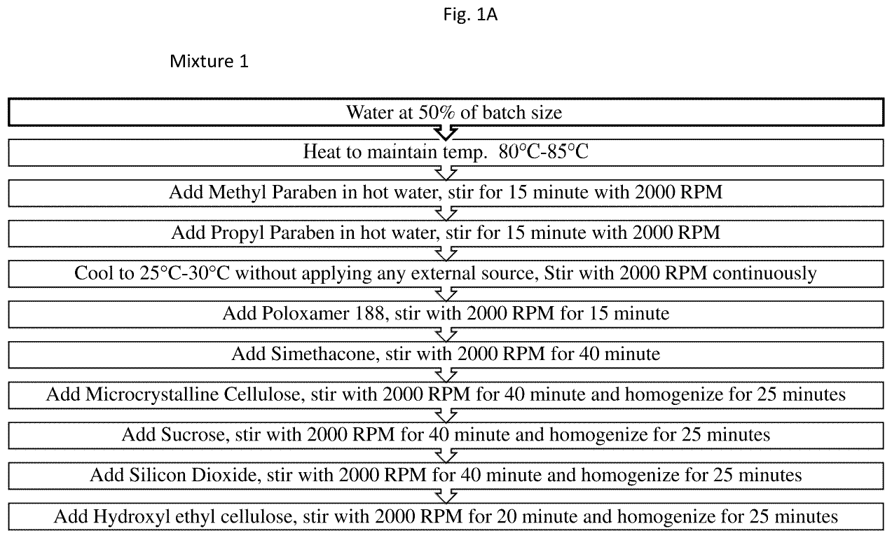

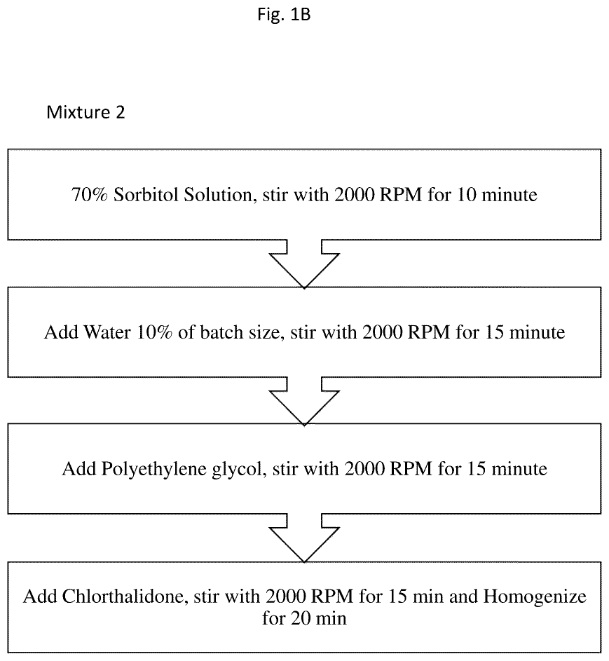

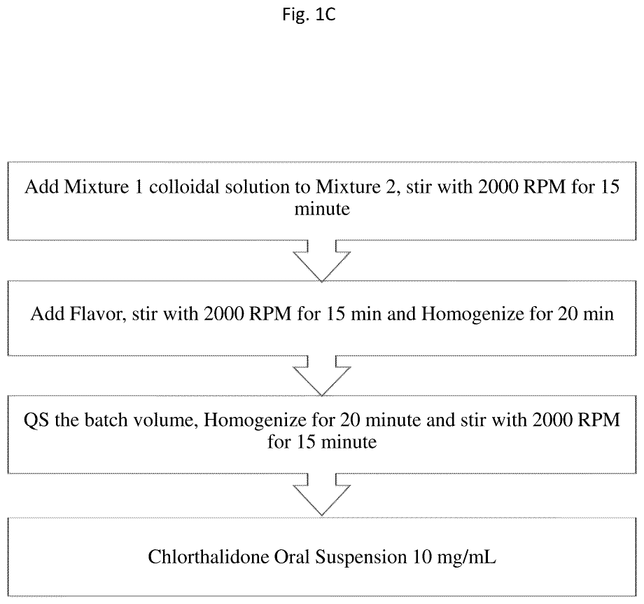

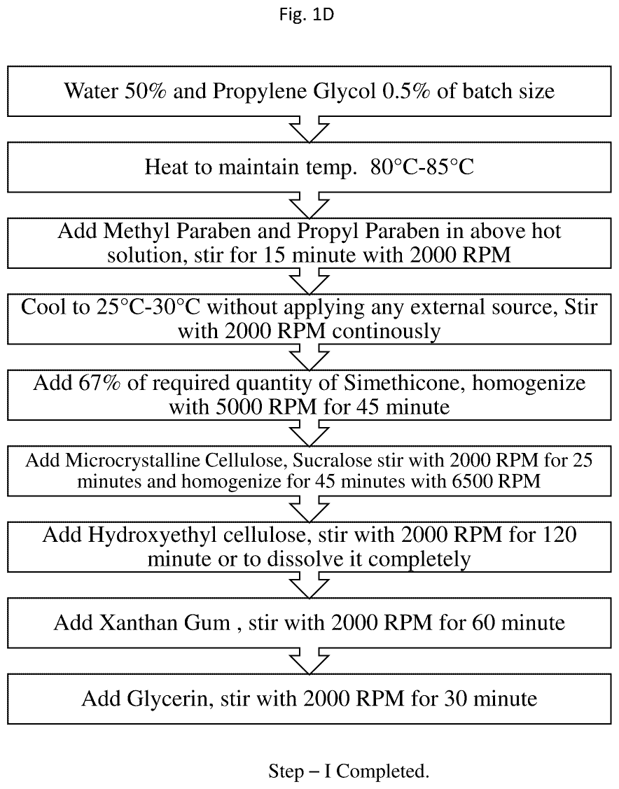

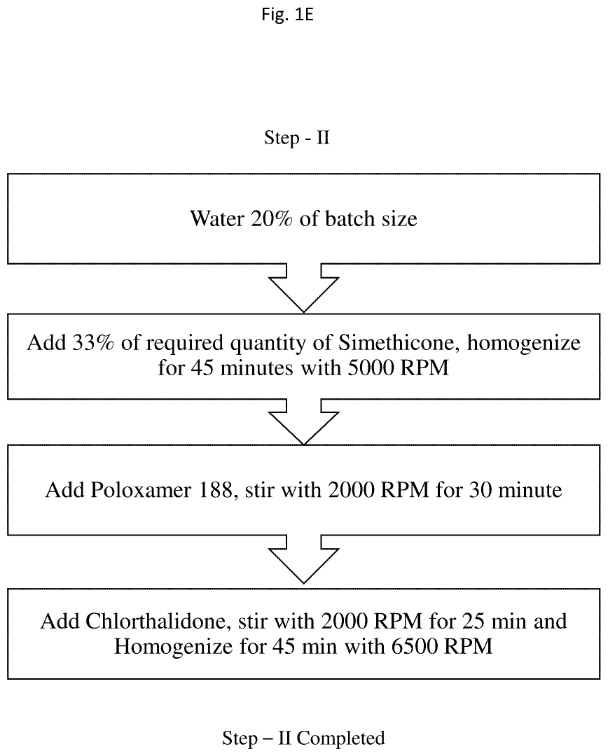

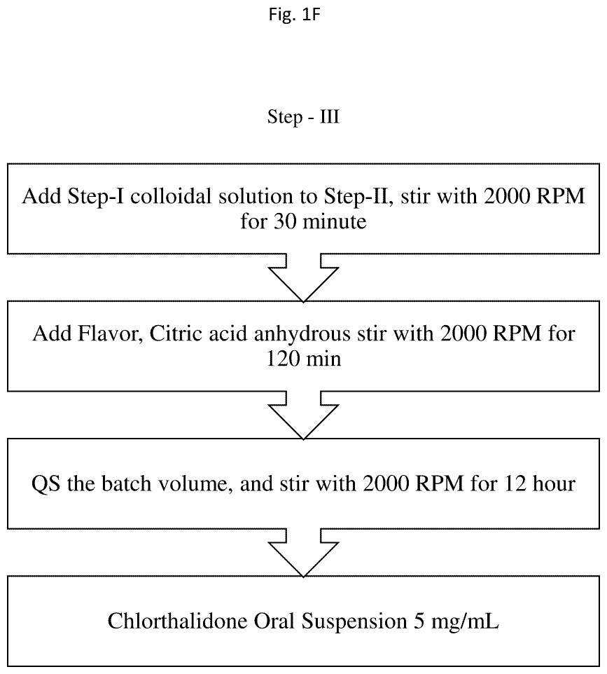

With reference to FIGS. 1A, 1B, and 1C, a preferred method (Process A) for making a stabilized chlorthalidone suspension includes making a first mixture by adding a solubilizing and/or a wetting agent to a first volume of water having a temperature of between about 25° C. to 30° C. with stirring, adding a suspending agent to the first volume of water with stirring and homogenizing, and adding at least one viscosity increasing agent or at least one anti-caking agent to the first volume of water with stirring and homogenizing. The first volume of water may be at or between 30% to 60% of the total end volume of the stabilized chlorthalidone suspension. For example, the first volume of water may be 30, 35, 40, 41, 42, 43, 44, 45, 46, 47, 48, 49 50, 51, 52, 52, 53, 54, 55, or 60% of the total volume (total batch volume) of the suspension.

The contemplated method also includes making a second mixture by adding a sweetening agent to a second volume of water with stirring, adding a viscosity increasing agent to the second volume of water with stirring, and adding chlorthalidone at between about 5 to 20 mg/mL to the second volume of water with stirring and homogenizing. The stabilized chlorthalidone suspension is formed by combining the first mixture and the second mixture with stirring to form a third mixture and homogenizing. In some embodiments, additional water may be added to bring the mixture to the calculated volume for the desired concentration of components including chlorthalidone.

In typical embodiments of any of the methods disclosed above or any method set forth below, the solubilizing and/or a wetting agent may be a wetting agent selected as one of poloxamer 188, poloxamer 124, or poloxamer 237. In more typical embodiments, the wetting agent is poloxamer 188. Preferably, the poloxamer 188 is added to the water or the first volume of water to have a final concentration of between about 5 mg/mL to about 20 mg/mL. Most preferably, the final concentration of the poloxamer 188 is 10 mg/mL.

In typical embodiments of any of the methods disclosed herein, the suspending agent is a polysaccharide suspending agent and/or a synthetic suspending agent. Examples of a polysaccharide suspending agent include microcrystalline cellulose (MCC), acacia gum, tragacanth gum, xanthan gums, starch, and/or alginates. Preferably, the suspending agent is MCC or MCC and xanthan gum added to the water or the first volume of water at a final concentration of between about 10 to 20 mg/mL. Most preferably, the final concentration of the MCC or MCC and xanthan gum are added to the water or the first volume of water is about 20 mg/mL.

In typical embodiments of any of the methods disclosed herein, the at least one viscosity increasing agent or the at least one anti-caking agent is hydroxyethylcellulose and/or silicon dioxide. Preferably, the at least one viscosity increasing agent or the at least one anti-caking agent is added to the water or the first volume of water to have a final concentration of between about 0.1 to 5% by weight (wt %).

In more preferred embodiments, the contemplated method for making the stabilized chlorthalidone suspension includes making the first mixture as disclosed above, wherein making the first mixture further includes first heating the water to about 80° C. to 85° C., adding at least one antimicrobial preservative to the heated water with stirring, and ambiently cooling the water to about 25° C. to 30° C., all of which occur prior to adding the solubilizing and/or the wetting agent. Preferably, the at least one antimicrobial is methyl paraben and/or propyl paraben. More preferably, the at least one antimicrobial is both methyl paraben and propyl paraben.

In additional preferred embodiments, making the first mixture further includes adding an anti-foaming agent to the first volume of water with stirring. Preferably the anti-foaming agent is simethicone. In other preferred embodiments, making the first mixture further includes adding a sweetening agent with stirring and homogenizing. Typically, the sweetening agent is sucrose, sucralose, stevia, and/or glycerin. Preferably, the sweetening agent is sucralose which is an acceptable sweetener for diabetics.

In still other preferred embodiments, the contemplated method of making a stabilized chlorthalidone suspension includes adding a flavor, a flavoring agent, a coloring agent, and/or a dye the water, or one of the first mixture, the second mixture, or the third mixture with stirring.

Preferably, the contemplated method of making a stabilized chlorthalidone suspension renders a suspension wherein a first concentration of the chlorthalidone in a first volume of the stabilized chlorthalidone differs from a second concentration in a second volume of the composition by 2.5% or less. More preferably, the first concentration of the chlorthalidone in a first volume of the stabilized chlorthalidone differs from a second concentration in a second volume of the composition by 1.0% or less.

With reference to FIGS. 1D, 1E, and 1F, a preferred method (Process B) for making a stabilized chlorthalidone suspension includes Steps I, II, and III. While Step I is described first, it would be understood that the solution mixture of Step II could be prepared first and then added to the solution mixture of Step I, or vice versa.

With reference to FIG. 1D (Step I of Process B), a method for making a stabilized chlorthalidone suspension includes a preparing a first solution mixture by adding a solubilizing and/or a wetting agent to a first volume of water having a temperature of between about 80° C. to 85° C. Preferably, the first volume of water is about 40% to 60% of the total batch volume of the final suspension, and more preferably, the first volume of water is 50% of the total batch volume of the final suspension. A solubilizing and/or a wetting agent is added to the first volume of water having a temperature of between about 80° C. to 85° C. Preferably, the solubilizing and/or the wetting agent is propylene glycol (PPG)(density of 1.04 g/ml). Typically, the amount of PPG is of or between about 0.20% to 1.0% of the total volume of the stabilized chlorthalidone suspension. More typically, the amount of PPG is of or between about 0.40% to 0.60% of the total volume of the stabilized chlorthalidone suspension. Most preferably, the amount of PPG is of or about 0.50% the total volume of the stabilized chlorthalidone suspension. In exemplary embodiments, the weight amount of PPG for a 5 mg/mL stabilized chlorthalidone suspension is 4.5 to 5.5 mg/mL. In more preferred embodiments, a 5 mg/mL stabilized chlorthalidone suspension includes PPG at 5.2 mg/mL.

With continued reference to FIG. 1D, a preservative is added to the heated water with PPG. Typically, the preservative is at least one of methyl paraben and propyl paraben, and more typically, both methyl paraben and propyl paraben are added. In the presence of PPG, the addition of both methyl paraben and propyl paraben may be sequential (e.g., one added immediately after the other), or each may be added simultaneously, and stirred. For sufficient mixing, the stirring may occur at 1,500 to 2,500 RPM for 10 to 15 minutes. Most preferably, the stirring occurs at 2,000 RPMS for 15 minutes. Methyl paraben may be present in the stabilized suspension at a concentration of between about 1 to 200 mg/mL, and propyl paraben may be present in the stabilized suspension at a concentration of between about 0.1 up to 40 mg/mL. Preferably, if both methyl paraben and propyl paraben are present in the suspension, an exemplary concentration of methyl paraben is about 1 mg/mL and an exemplary concentration of propyl paraben is about 0.2 mg/mL.

With continued reference to FIG. 1D, after the addition of a preservative (e.g., methyl paraben and/or propyl paraben) to the heated water and PPG solution, the solution is allowed to cool to 25° C. to 30° C. with removal of heat, and without applying any external source. Typically, stirring (e.g., at or between 1,500 to 2,500 RPM) continues during the cooling from 80° C. to 85° C. down to 25° C. to 30° C. In some preferred embodiments, potassium sorbate is added to the water solution of PPG and at least one of methyl paraben and propyl paraben after or upon cooling to 25° C. to 30° C. More preferably, both methyl paraben and propyl paraben are added to the water and PPG solution at 80° C. to 85° C., and potassium sorbate is added after or upon cooling (with removal of heat, and without applying any external source) to 25° C. to 30° C. with stirring.

With continued reference to FIG. 1D, once the water solution with PPG and preservative(s) is at a temperature of 25° C. to 30° C., and optionally potassium sorbate added, an anti-foaming agent is added to the solution. Preferably, the anti-foaming agent is simethicone. In order to effectively mix the simethicone into the solution, 50% to 75% of the total final concentration of simethicone is added to the solution. More typically, 60% to 70% of the final concentration of simethicone is added to the solution, and preferably 67% of the final concentration of simethicone is added. For uniform dispersion of the simethicone, the solution is homogenized at 4,500 to 5,500 RPM for 30 to 50 minutes. Preferably, the solution is homogenized at 5,000 RPM for 45 minutes. In exemplary embodiments, simethicone is present in the stabilized suspension at a final concentration of between about 5 mg/mL to about 9 mg/mL. More preferably, simethicone is present at a final concentration between about 8 mg/mL to about 9 mg/mL.

With continued reference to FIG. 1D, after the simethicone is dispersed in the solution, microcrystalline cellulose (MCC) and a sweetening agent (e.g., sucralose, sucrose, glycerin, stevia, and/or sorbitol) are added with stirring either simultaneously or sequentially (e.g., one immediately after the other). Typically, the stirring is at 1,500 to 2,500 RPM for 15 to 25 minutes. In addition to or alternative to the stirring, the MCC and sweetening agent are homogenized in the solution at 6,000 to 7,000 RPM for about 40 to 50 minutes. Preferably, the homogenization occurs at 6,500 RPM for about 45 minutes. More preferably, the homogenization is carried out in addition to the stirring to thoroughly disperse the MCC. Most preferably, the sweetening agent is sucralose which at effective amounts is readily soluble and does not require additional time for dispersion. Typically, the MCC is present in the stabilized suspension at a final concentration of between about 10 to 20 mg/mL. More typically, the MCC is present in the stabilized suspension at a concentration of 20 mg/mL.

With continued reference to FIG. 1D, an anti-caking agent is added with stirring to the aqueous solution of PPG, preservative(s), simethicone, MCC, and a sweetening agent. The anti-caking agent is preferably hydroxyethylcellulose (HEC). The addition of an anti-caking agent like HEC increases the viscosity of the solution, and while both stirring and homogenization can effectively dissolve the HEC completely, homogenization may result in the formation of bubbles which is not desired. Typically, stirring is carried out at 1,500 to 2,500 RPM for 90 minutes (1.5 hours) to 150 minutes (2.5 hours). More typically, stirring is carried out at 2,000 RPM for 120 minutes (2 hours). Alternatively, while homogenization could cause the formation of bubbles, the addition of the remaining amount of simethicone added in Step II of Process B, will remove the bubbles.

With continued reference to FIG. 1D, a viscosity increasing agent is added with stirring to the aqueous solution of PPG, preservative(s) (e.g., methyl paraben, propyl paraben, and/or potassium sorbate), simethicone, MCC, the sweetening agent (e.g., sucralose), and the anti-caking agent (e.g., HEC). Preferably, the viscosity increasing agent is xanthan gum. The addition and dissolution of the viscosity increasing agent like xanthan gum is carried out with stirring. Typically, effective stirring for dissolution of the viscosity increasing agent (e.g., xanthan gum) is at 1,500 to 2,500 RPM for 40-80 minutes. More typically, effective stirring for dissolution of the viscosity increasing agent (e.g., xanthan gum) occurs at 2,000 RPM for 60 minutes (1 hour).

With continued reference to FIG. 1D, in the last step of Step I, glycerin is added with stirring to the aqueous solution of PPG, preservative(s) (e.g., methyl paraben and propyl paraben), simethicone, MCC, the sweetening agent (e.g., sucralose), the anti-caking agent (e.g., HEC), and the viscosity increasing agent (e.g., xanthan gum). Glycerin in this solution is a sweetener, a viscosity increasing agent, and an antimicrobial agent. By increasing the viscosity, glycerin reduces the sedimentation rate of the final suspension. The addition and dissolution of glycerin is carried out with stirring. Typically, effective stirring for dissolution of glycerin is at 1,500 to 2,500 RPM for 20-40 minutes. More typically, effective stirring for dissolution of glycerin occurs at 2,000 RPM for 30 minutes.

With reference to FIG. 1E, (Step II of Process B), a method for making a stabilized chlorthalidone suspension also includes preparing a second solution mixture starting with a volume of water (e.g., a second volume of water distinct and separate from the volume of water in Step I). This second volume of water is preferably between about 10% to 30% of the total volume of the final suspension. More preferably, the second volume of water is 20% of the total volume of the final suspension (e.g., the total batch size). To this volume of water, the remaining volume of simethicone is added relative to the amount added in Step I. Accordingly, if 50% to 75% of the total final concentration of simethicone is added in Step I, then a corresponding 25% to 50% is added to the second volume of water. More typically, 30% to 40% of the final concentration of simethicone is added to the second volume of water, and preferably 33% of the final concentration of simethicone is added to the second volume of water if 67% of the final concentration was added in Step 1. Simethicone is used as an anti-foaming agent to prevent foam formation upon addition of a solubilizing and/or a wetting agent (e.g., Poloxamer 188). The simethicone is mixed into the second volume of water by homogenization. Typically, homogenization occurs at or between 4,500 to 5,500 RPM for 40 to 50 minutes. More typically, homogenization of the simethicone occurs at 5,000 RPM for 45 minutes.

With continued reference to FIG. 1E, a solubilizing and/or a wetting agent is added to the second water mixture and simethicone. Preferably, the solubilizing and/or a wetting agent is a wetting agent such as poloxamer 188. Typically, poloxamer 188 is dispersed into the second water mixture with simethicone by stirring at 1,800 to 2,200 RPM for 20 to 40 minutes. More typically, poloxamer 188 is dispersed into the second water mixture with simethicone by stirring at 2,000 RPM for 30 minutes.

With continued reference to FIG. 1E, chlorthalidone is added to the second water mixture of simethicone and a solubilizing and/or a wetting agent. Preferably, chlorthalidone is added at 5 mg/mL final concentration for the final volume/batch size. The chlorthalidone is added with stirring at 1,800 to 2,200 RPM for 20 to 30 minutes followed by homogenization at 6,000 to 7,000 RPM for 40 to 50 minutes. More preferably, chlorthalidone is added at 5 mg/mL final concentration with stirring at 2,000 RPM for 25 minutes followed by homogenization at 6,500 RPM for 45 minutes.

With reference to FIG. 1F, the solutions mixtures from Step I and Step II are combined and mixed. Typically, the colloidal solution of Step I is added to the solution of Step II. The combined solutions are mixed with stirring at 1,800 to 2,200 RPM for 20 to 40 minutes. Preferably the Step I and Step II solutions are mixed with stirring at 2,000 RPM for 30 minutes.

With reference to FIG. 1F, a buffering agent is added to the combined solutions, which upon mixing forms a stabilized chlorthalidone suspension (without added flavoring or color). Preferably, the buffering agent is citric acid anhydrous added at or between about 0.02 to 0.05 mg/mL. Preferably, citric acid anhydrous is added at 0.034 mg/mL. Subsequently, (e.g., one immediately added after the other) or simultaneously, a flavor, a flavoring agent, a coloring agent, and/or a dye, is added to the water solution, and is mixed with stirring for at 1,800 to 2,200 RPM for 90 minutes (1.5 hours) to 150 minutes (2.5 hours). Preferably, the stirring is at 2,000 RPM for 120 minutes (2 hours).

With continued reference to FIG. 1F, with the buffering agent (e.g., citric acid anhydrous) with or without a flavor, a flavoring agent, a coloring agent, and/or a dye dispersed into solution, the solution may be sampled and tested for quality control. For quality control testing, the chlorthalidone suspension may be tested for the concentration of one or more components. Typically, the chlorthalidone suspension is tested to confirm that a first concentration of the chlorthalidone in a first volume of the composition differs from a second concentration in a second volume of the composition by no more than 5%, as described in more detail herein. In other words, the chlorthalidone suspension is at least 95% homogenous with respect to the concentration of chlorthalidone found throughout the dispersion. Preferably, the chlorthalidone suspension is at least 95% homogenous with respect to all components throughout the suspension.

With continued reference to FIG. 1F, with the quality control sample or samples confirmed, the solution is then stirred at 1,500 to 2,500 RPM for 2 to 15 hours. Preferably, the verified solution is stirred at 2,000 RPM for 8 to 12 hours.

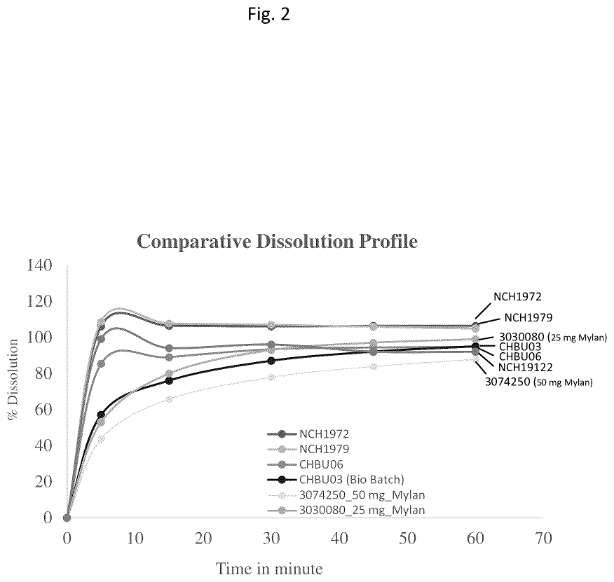

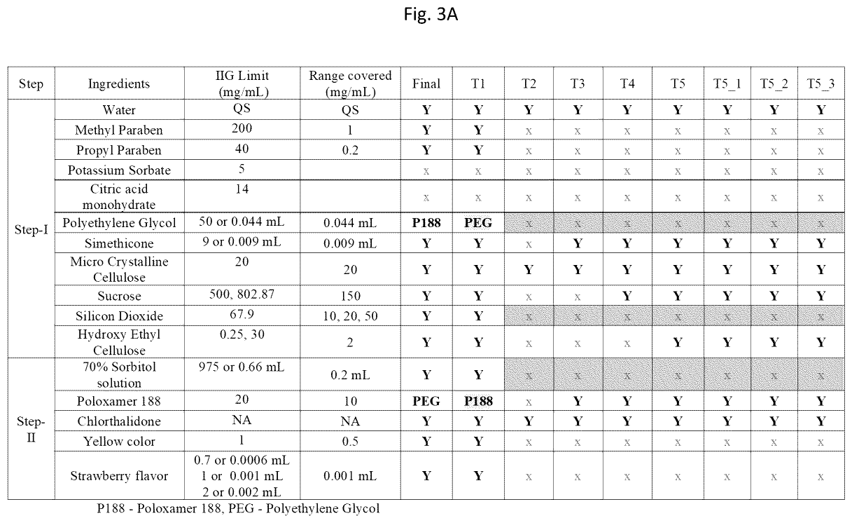

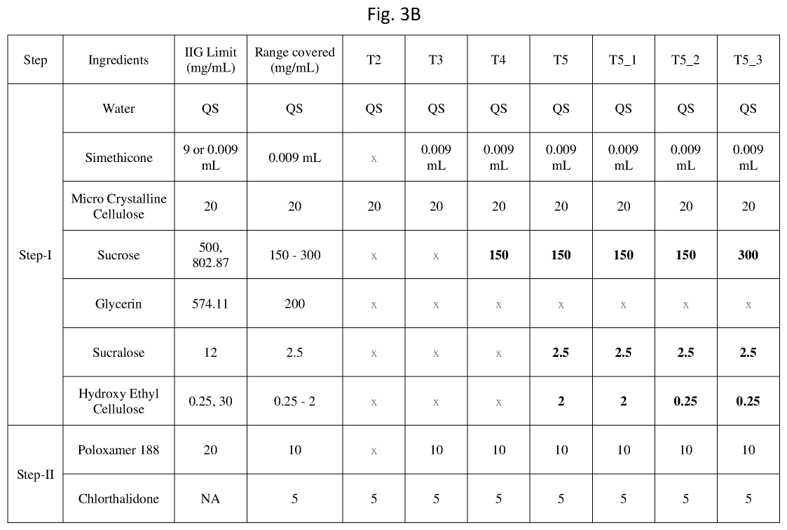

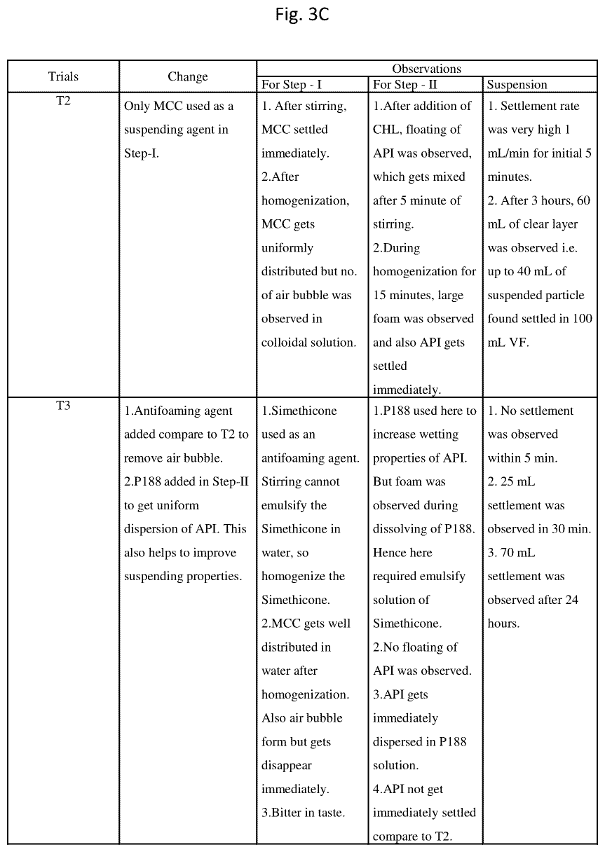

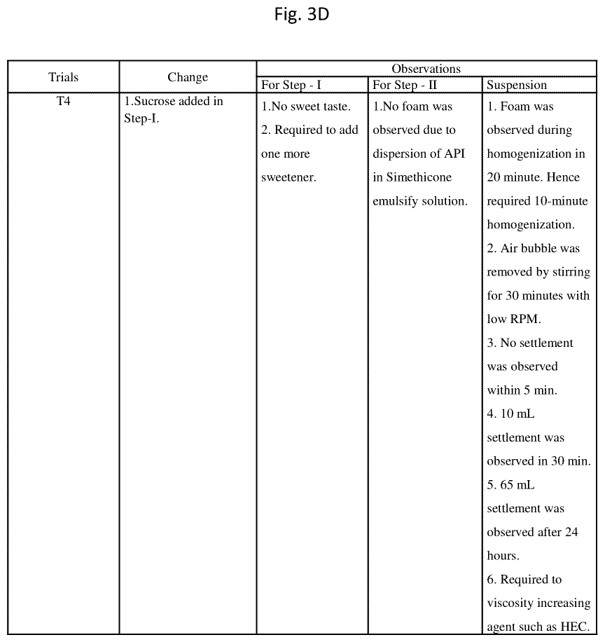

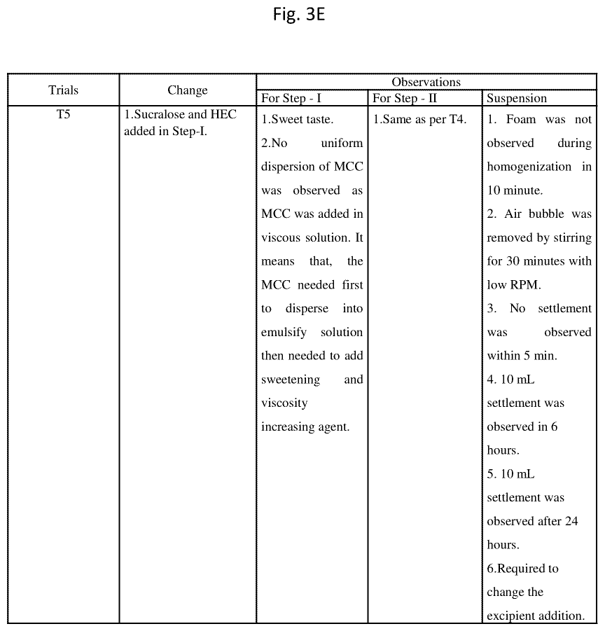

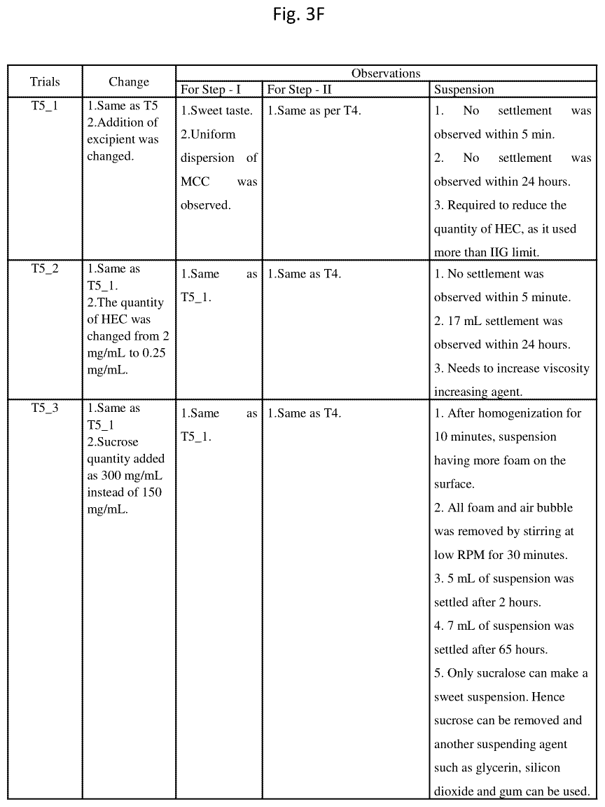

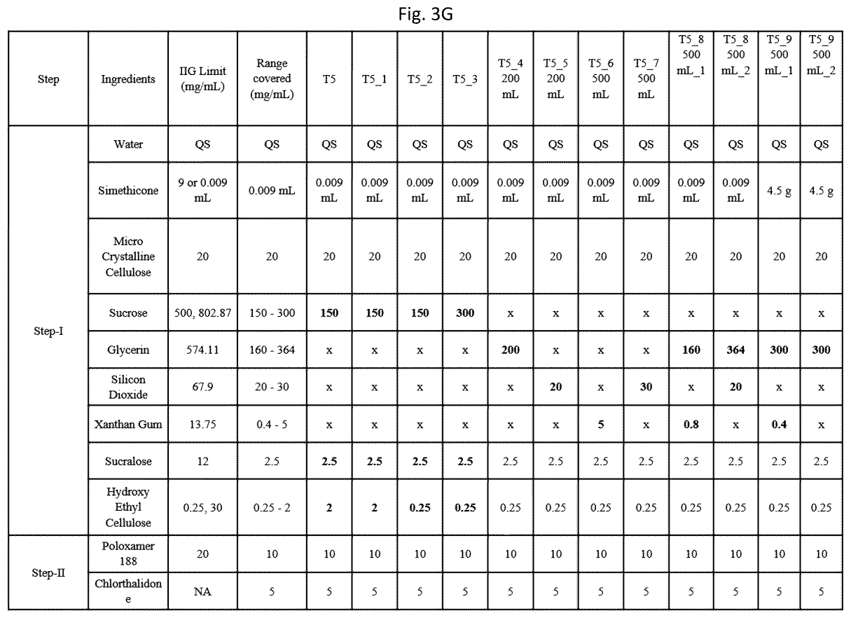

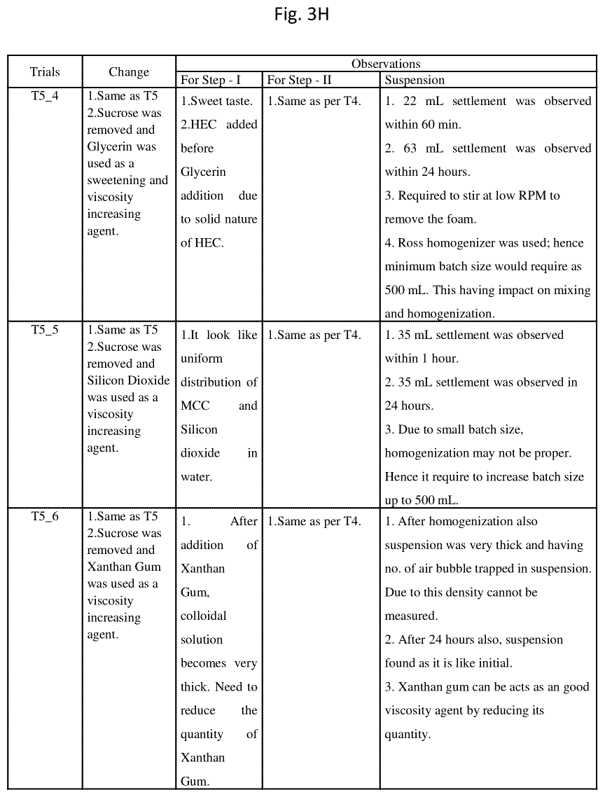

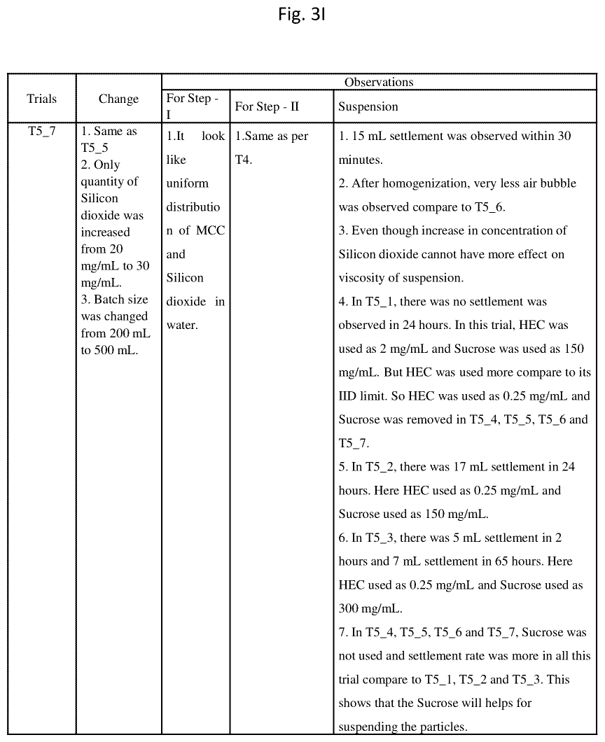

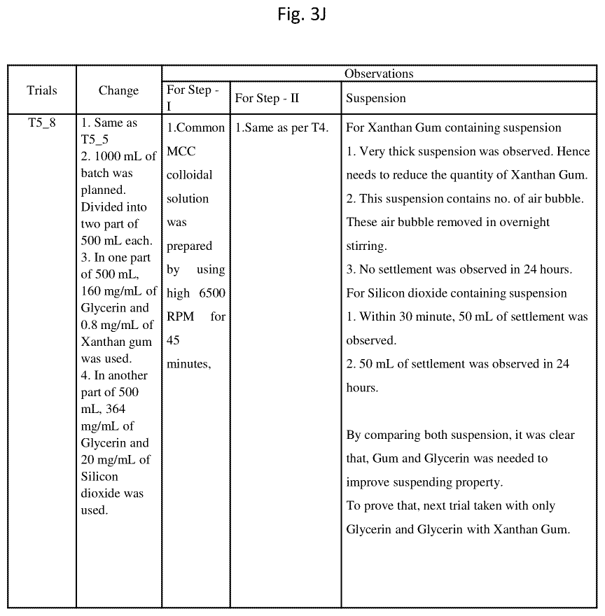

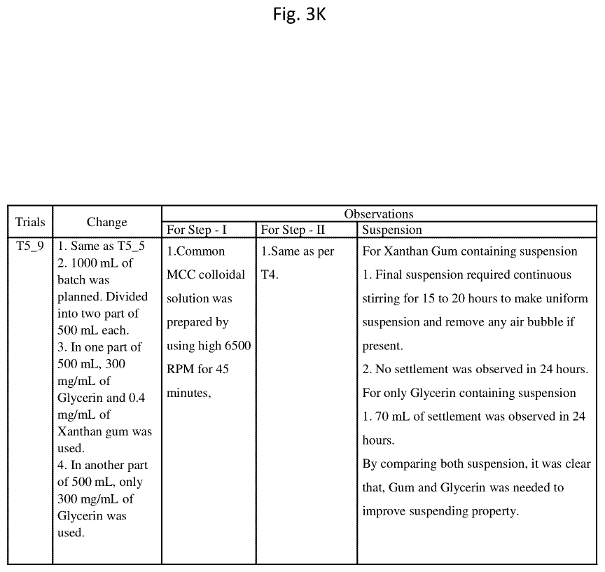

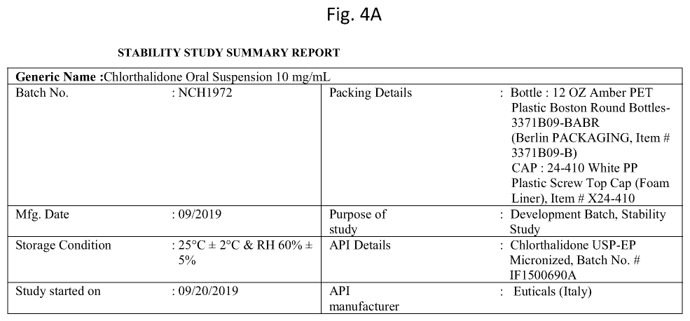

With reference to FIG. 2 and FIGS. 3A-3K, exemplary results show comparable data of various concentrations and the effects of the presently disclosed chlorthalidone dispersion with and without poloxamer and with and without hydroxyethylcellulose. Additionally, these results also show data for the chlorthalidone dispersion with and without simethicone, sucrose, silicon dioxide, and sorbitol, as well as flavors and colors as indicated (FIGS. 3A-3K).

### EXAMPLES

With reference to Table 1A below, three exemplary chlorthalidone suspension formulations (Formulation 1, Formulation 2, and Formulation 3), were prepared following Process A as disclosed herein. Exemplary excipients including their function and IIG limit are indicated along with corresponding concentrations for each. As indicated, the amount of silicon dioxide varies for each of the formulations.

TABLE 1A
Formulation Composition (Process A)

| Active and | IIG Limit | | Formulation-1 | Formulation-2 | Formulation-3 |
|---|---|---|---|---|---|
| Excipient Details | (mg/mL) | Function | (mg/mL) | (mg/mL) | (mg/mL) |
| Chlorthalidone | API | Active | 10 | 10 | 10 |
| Water for Injection/ | Q.S. to mL | Vehicle | Q.S. to mL | Q.S. to mL | Q.S. to mL |
| Deionized Water | | | | | |
| Methyl Paraben | 200 | Antimicrobial | 1 | 1 | 1 |
| | | Preservative | | | |
| Propyl Paraben | 40 | Antimicrobial | 0.2 | 0.2 | 0.2 |
| | | Preservative | | | |
| Poloxamer 188 | 20 | Emulsifying/Wetting | 10 | 10 | 10 |
| | | Agent | | | |
| Simethicone | 9 | Antifoaming | 0.009 mL | 0.009 mL | 0.009 mL |
| (Density: 0.971) | | Agent | | | |
| Microcrystalline | 20 | Suspending Agent | 20 | 20 | 20 |
| Cellulose (Avicel | | | | | |
| pH-101) | | | | | |
| Sucrose | 500, 802.87 | Sweetening Agent | 150 | 150 | 150 |
| Silicon Dioxide | 67.94 | Viscosity- | 50 | 10 | 20 |
| | | Increasing Agent | | | |
| Hydroxyethyl | 0.25, 30 | Viscosity- | 2 | 2 | 2 |
| cellulose | | Increasing Agent, | | | |
| | | Anticaking Agents | | | |
| Sorbitol F Solution | 975 | Sweetening Agent | 0.2 mL | 0.2 mL | 0.2 mL |
| 70% in Water | | | | | |
| (Density: 1.46) | | | | | |
| Polyethylene | 50 | Viscosity- | 0.044 mL | 0.044 mL | 0.044 mL |
| Glycol 400 | | Increasing Agent, | | | |
| (Density: 1.13) | | Antioxidant Agent | | | |
| Strawberry Flavor | 0.7 or | Flavor | 0.001 mL | 0.001 mL | 0.001 mL |
| ART WS | 0.0006 mL, | | | | |
| (Density: 1.03) | 1 or 0.001 mL | | | | |
| FD & C Yellow # | 1* | Color | 0.5 | 0.5 | 0.5 |
| 5/Tartrazine AL | | | | | |
| 15%-17% | | | | | |

*Maximum usage is not known.

With reference to Table 1B, the Formulations 1, 2, and 3 of Table 1A prepared by Process A are compared to the components for a stabilized chlorthalidone suspension prepared by Process B as disclosed herein and set forth in FIGS. 1D, 1E, and 1F.

TABLE 1B
Formulation Composition Comparison of Process A with Process B
Process A

| Active and Excipient | IIG Limit | | Formulation-1 | Formulation-2 | Formulation-3 |
|---|---|---|---|---|---|
| Details | (mg/mL) | Function | (mg/mL) | (mg/mL) | (mg/mL) |
| Chlorthalidone | API | Active | 10 | 10 | 10 |
| Water for Injection/ | Q.S. to mL | Vehicle | Q.S. to mL | Q.S. to mL | Q.S. to mL |
| Deionized Water | | | | | |
| Methyl Paraben | 200 | Antimicrobial | 1 | 1 | 1 |
| | | Preservative | | | |
| Propyl Paraben | 40 | Antimicrobial | 0.2 | 0.2 | 0.2 |
| | | Preservative | | | |
| Poloxamer 183 | 20 | Wetting Agent | 10 | 10 | 10 |
| Simethicone | 9 | Antifoaming Agent | 0.009 mL | 0.009 mL | 0.009 mL |
| (Density: 0.971) | | | | | |
| Microcrystalline | 20 | Suspending Agent | 20 | 20 | 20 |
| Cellulose (Avicel | | | | | |
| pH-101) | | | | | |
| Sucrose | 500. 802.87 | Sweetening Agent | 150 | 150 | 150 |
| Silicon Dioxide | 67.94 | Viscosity-Increasing | 50 | 10 | 20 |
| | | Agent | | | |
| Hydroxyethyl cellulose | 0.25. 30 | Viscosity-Increasing | 2 | 2 | 2 |
| | | Agent. Anticaking | | | |
| | | Agents | | | |
| Sorbitol F Solution 70% | 975 | Sweetening Agent | 0.2 mL | 0.2 mL | 0.2 mL |
| in Water (Density: 1.46) | | | | | |
| Polyethylene Glycol 400 | 50 | Viscosity-Increasing | 0.044 mL | 0.044 mL | 0.044 mL |
| (Density: 1.13) | | Agent. | | | |
| | | Antioxidant Agent | | | |
| Strawberry Flavor ART | 0.7 or 0.0006 mL. | Flavor | 0.001 mL | 0.001 mL | 0.001 mL |
| WS (Density: 1.03) | 1 or 0.001 mL | | | | |
| FD & C Yellow # 5/ | 1* | Color | 0.5 | 0.5 | 0.5 |
| Tartrazine AL 15%-17% | | | | | |

Process B

| Active and | IIG Limit | | Formulation |
|---|---|---|---|
| Excipient Details | (mg/mL) | Function | (mg/mL) |
| Chlorthalidone | API | Active | 5 |
| Deionized Water | Q.S. to mL | Vehicle | Q.S. to mL |
| Methyl Paraben | 200 | Antimicrobial | 1 |
| | | Preservative | |
| Propyl Paraben | 40 | Antimicrobial | 0.2 |
| | | Preservative | |
| Poloxamer 188 | 20 | Wetting | 10 |
| | | Agent | |
| Simethicone | 9 | Antifoaming | 9 |
| (Density: 0.971) | | Agent | |
| Microcrystalline | 20 | Suspending | 20 |
| Cellulose | | Agent | |
| (Avicel pH-101) | | | |
| Sucralose | 12 | Sweetening | 2.6 |
| | | Agent | |
| Xanthan Gum | 13.75 | Viscosity- | 0.4 |
| | | Increasing | |
| | | Agent | |
| Hydroxyethyl | 0.25. 30 | Viscosity- | 0.25 |
| cellulose | | Increasing | |
| | | Agent. | |
| | | Anticaking | |
| | | Agents | |
| Glycerin | 574.11 | Sweetening | 300 |
| | | Agent | |
| Propylene Glycol | 180 | Viscosity- | 0.005 mL |
| | | Increasing | |
| | | Agent. | |
| | | Antioxidant | |
| | | Agent | |
| Orange | 2.4. 10. 12 | Flavor | 0.6 μL/mL |
| Citric acid | 15 | Buffering | 0.034 |
| anhydrous | | agent | |

With reference to Table 2, each of Formulation 1, 2, and 3 was prepared as a 500 ml (Formulation 1) or 1,000 ml batch size (Formulation 2 and 3) following the methods disclosed herein and set forth in FIGS. 1A, 1B, and 1C, with 50, 10, or 20 mg/mL silicon dioxide as indicated, with observations noted (Remarks).

TABLE 2
Batch Details
Formulation trials of Chlorthalidone Oral Suspension 10 mg/vial

| | Formulation 1 | | | | Formulation 2 | | | | Formulation 3 | | | | |
|---|---|---|---|---|---|---|---|---|---|---|---|---|---|
| Active and | B.No.# NCH1972 | | | | B.No.# NCH1979 | | | | B.No.# NCH1984 | | | | |
| Excipient | Batch Size: 500 mL | | | | Batch Size: 1000 mL | | | | Batch Size: 1000 mL | | | | |
| Details | mg/mL | | Batch Qty. | | mg/mL | | Batch Qty. | | mg/ml | | Batch Qty. | | Remarks |
| Chlorthalidone | 10 | | 5 g or | | 10 | | 10 g or | | 10 | | 10 g or | | In Formulation |
| | | | 5000 mg | | | | 10000 mg | | | | 10000 mg | | 1, more lumps |
| Water for | Q.S. to | | Q.S. to 500 | | Q.S. to | | Q.S. to 1000 | | Q.S. to | | Q.S. to 1000 | | are observed |
| Injection/ | mL | | mL | | mL | | mL | | mL | | mL | | after addition |
| Deionized | | | | | | | | | | | | | of Silicon |
| Water | | | | | | | | | | | | | dioxide. So two |
| Methyl Paraben | 1 | | 500 | mg | 1 | | 1000 | mg | 1 | | 1000 | mg | more batches |
| Propyl Paraben | 0.2 | | 100 | mg | 0.2 | | 200 | mg | 0.2 | | 200 | mg | manufactured |
| Poloxamer 188 | 10 | | 5000 mg or | | 10 | | 10000 mg or | | 10 | | 10000 mg or | | using less |
| | | | 5 g | | | | 10 g | | | | 10 g | | quantity of |
| Simethicone | 0.009 | mL | 4.5 | mL | 0.009 | mL | 9 | mL | 0.009 | mL | 9 | mL | Silicon dioxide. |
| (Density: 0.971) | | | | | | | | | | | | | Formulation 3 |
| Microcrystalline | 20 | | 10000 mg | | 20 | | 20000 mg or | | 20 | | 20000 mg or | | found suitable |
| Cellulose | | | or 10 g | | | | 20 g | | | | 20 g | | as a suspension |
| (Avicel pH-101) | | | | | | | | | | | | | compare to |
| Sucrose | 150 | | 75000 mg | | 150 | | 150000 mg or | | 150 | | 150000 mg or | | Formulation |
| | | | or 75 g | | | | 150 g | | | | 150 g | | 2 which look |
| Silicon Dioxide | 50 | | 25000 mg | | 10 | | 10000 mg or | | 20 | | 20000 mg or | | like a low |
| | | | or 25 g | | | | 10 g | | | | 20 g | | viscous and |
| Hydroxyethyl | 2 | | 1000 mg or | | 2 | | 2000 mg or | | 2 | | 2000 mg or | | low dense |
| cellulose | | | 1 g | | | | 2 g | | | | 2 g | | suspension. |
| Sorbitol F | 0.2 | mL | 100 | mL | 0.2 | mL | 200 | mL | 0.2 | mL | 200 | mL | |
| Solution 70% in | | | | | | | | | | | | | |
| Water | | | | | | | | | | | | | |
| (Density: 1.46) | | | | | | | | | | | | | |
| Polyethylene | 0.044 | mL | 22 | mL | 0.044 | mL | 44 | mL | 0.044 | mL | 44 | mL | |
| Glycol 400 | | | | | | | | | | | | | |
| (Density: 1.13) | | | | | | | | | | | | | |
| Strawberry | 0.001 | mL | 0.5 | mL | 0.001 | mL | 1 | mL | 0.001 | mL | 1 | mL | |

| | | | | | | |
|---|---|---|---|---|---|---|
| Flavor ART WS | | | | | | |
| (Density: 1.03) | | | | | | |
| FD & C Yellow | 0.5 | 250 mg or | 0.5 | 500 mg or | 0.5 | 500 mg or |
| #5/Tartrazine | | 0.25 g | | 0.5 g | | 0.5 g |
| AL 15%-17% | | | | | | |

With reference to Table 3, dissolution of chlorthalidone in a suspension pursuant to Formulation 1 (NCH1972) and Formulation 2 (NCH1979) was compared to the dissolution of 50 mg tablets of chlorthalidone as indicated. Notably, with reference to FIG. 2, the dissolution of Formulation 1 and Formulation 2 occurred within 5 minutes and was maintained for at least 1 hour (60 minutes). Comparatively, the 50 mg tablets did not disperse or dissolve as quickly, and not as much of the chlorthalidone went into solution after 1 hour.

TABLE 3
Comparative Dissolution Profile Tablet Vs Suspension

| | Product Name: | | | | | | |
|---|---|---|---|---|---|---|---|
| | | | Chlorthalidone | | | | |
| | | | Oral | Chlorthalidone | Chlorthalidone | Chlorthalidone | Chlorthalidone |
| | Chlorthalidone Oral | | Suspension 5 | Tablets | Tablets | Tablets | Tablets |
| | Suspension 10 mg/ml | | mg/ml | 50 mg | 50 mg | 50 mg | 50 mg |
| Batch No.: | NCH1972 | NCH1979 | NCH19122 | CHBU06 | CHBU03 | 3074250_50 | 3030080_25 |
| | | | | | (Bio Batch) | mg_Mylan | mg_Mylan |
| Condition: | Initial | Initial | Initial | Initial | Initial | Initial | Initial |

Dissolution Condition:

| Apparatus | USP Type II | USP Type II | USP Type II | USP Type II | USP Type II | USP Type II | USP Type II |
|---|---|---|---|---|---|---|---|
| Volume | 900 mL | 900 mL | 900 mL | 900 mL | 900 mL | 900 mL | 900 mL |
| RPM | 75 RPM | 75 RPM | 75 RPM | 75 RPM | 75 RPM | 75 RPM | 75 RPM |
| Temperature | 37.0 ± 0.5° C. | 37.0 ± 0.5° C. | 37.0 ± 0.5° C. | 37.0 ± 0.5° C. | 37.0 ± 0.5° C. | 37.0 ± 0.5° C. | 37.0 ± 0.5° C. |
| Sampling | 5, 15, 30, | 5, 15, 30, | 5, 15, 30, | 5, 15, 30, | 5, 15, 30, | 5, 15, 30, | 5, 15, 30, |
| point in | 45, 60 | 45, 60 | 45, 60 | 45, 60 | 45, 60 | 45, 60 | 45, 60 |
| minute | | | | | | | |
| Time in | % of | % of | % of | % of | % of | % of | % of |
| minute | dissolution | dissolution | dissolution | dissolution | dissolution | dissolution | dissolution |
| 0 | 0 | 0 | 0 | 0 | 0 | 0 | 0 |
| 5 | 106 | 109 | 99 | 85 | 57 | 44 | 53 |
| 15 | 106 | 108 | 94 | 89 | 76 | 66 | 80 |
| 30 | 106 | 107 | 96 | 93 | 87 | 78 | 93 |
| 45 | 106 | 106 | 92 | 94 | 92 | 84 | 97 |
| 60 | 106 | 105 | 92 | 95 | 95 | 88 | 99 |

With reference to Table 4, each of Formulation 1, 2 and 3 was analyzed for settling. In other words, the concentration of each of chlorthalidone, methyl paraben, and propyl paraben was analyzed throughout the suspension in order to determine the degree of dispersion of each in the suspension. Accordingly, for each of three suspensions, two samples were taken from the bottom, the middle and the top of the suspension and the concentration of chlorthalidone, methylparaben, and propylparaben were quantified for each sample. As indicated, the concentration of chlorthalidone in each of the samples varied (i.e., differed) by no more than 1%.

TABLE 4
Analytical Results

| | | Batch No.: NCH1972 | | | Batch No.: NCH1979 | | | Batch No.: NCH1984 | | |
|---|---|---|---|---|---|---|---|---|---|---|
| Analytical | | Chlor- | Methyl | Propyl | Chlor- | Methyl | Propyl | Chlor- | Methyl | Propyl |
| Data | Samples | thalidlone | Paraben | Paraben | thalidlone | Paraben | Paraben | thalidlone | Paraben | Paraben |

| | | | | | | | | | | |
|---|---|---|---|---|---|---|---|---|---|---|
| Assay | Bottom-1 | 106.3 | 101.7 | 99.8 | 104.5 | 102.3 | 98.7 | 99.9 | 101.2 | 98.2 |
| | Bottom-2 | 106.9 | 102.2 | 100.7 | 104.1 | 102.3 | 99.4 | 100.2 | 101.4 | 98.3 |
| | Middle-1 | 107.0 | 102.1 | 100.2 | 102.1 | 100.1 | 97.2 | 100.0 | 101.3 | 98.0 |
| | Middle-2 | 106.4 | 102.0 | 99.9 | 101.9 | 100.0 | 97.0 | 100.3 | 101.5 | 98.4 |
| | Top-1 | 106.5 | 101.9 | 99.6 | 101.8 | 100.2 | 97.2 | 99.4 | 100.7 | 97.5 |
| | Top-2 | 106.5 | 101.7 | 99.6 | 101.9 | 99.9 | 97.2 | 99.7 | 101.0 | 97.8 |

| Observation | Batch QS made with volume. | | | NA | | Batch QS made with weight. | |
|---|---|---|---|---|---|---|---|
| pH | 5.21 | | | 5.34 | | 5.54 | |
| Density | 1.1764 | | g cm3 | 1.12941 | g cm3 | 1.17897 | g cm3 |
| Viscocity | 290 | | cp | 260 | cp | 250 | ep |
| Deliverable | NA | | | NA | | Complies as per USP <698> | |
| volume | | | | | | for multi unit container for | |
| | | | | | | not less than 100% LV | |
| Dissolution | Sample-1 | 106 | Not | 107 | Not | 102 | Not |
| (75 RPM, | Sample-2 | 106 | Applicable | 108 | Applicable | 106 | Applicable |
| Water, 30 | Sample-3 | 106 | | 107 | | 107 | |
| minutes) | Sample-4 | 106 | | 107 | | 108 | |
| | Sample-5 | 105 | | 107 | | 105 | |
| | Sample-6 | 105 | | 106 | | 103 | |
| | Mean | 106 | | 107 | | 105 | |

With reference to Table 5, each of Formulations 1, 2, and 3 (NCH1972, NCH1979, NCH1984) were analyzed for impurities.

TABLE 5
Impurity Data

| | | B. No.# IF1500690A |
|---|---|---|
| Impurity Name | Limit | (Euticals API) |
| Imp-B/Chlorthalidone | NMT 0.7% | 0.28 |
| Related Compound-A | | |
| Imp-J | NMT 0.3% | 0.01 |
| Imp-G | NMT 0.2% | 0.10 |
| Any unspecified | NMT 0.1% | 0.05 |
| impurity | | |
| Total Impurity | NMT 1.0% | 0.48 |

| | | B. No. # NCH1972 | | B. No. # NCH1979 | B. No. # NCH1984 |
|---|---|---|---|---|---|
| Impurity Name | Limit | Sample-1 | Sample-2 | Sample-1 | Sample-1 |
| Imp-A | NMT 0.7% | 0.45 | 0.45 | 0.41 | 0.39 |
| Imp-J | NMT 0.2% | 0.02 | 0.02 | 0.01 | 0.00 |
| Imp-G | NMT 0.2% | 0.11 | 0.12 | 0.11 | 0.11 |
| CPSP | NMT 0.10% | ND | ND | ND | ND |
| Any Individual | NMT 0.20% | 0.05 | 0.05 | 0.01 | 0.01 |
| Total Imp | NMT 0.80% | 0.18 | 0.19 | 0.13 | 0.12 |
| [Excluding Imp-A] | | | | | |

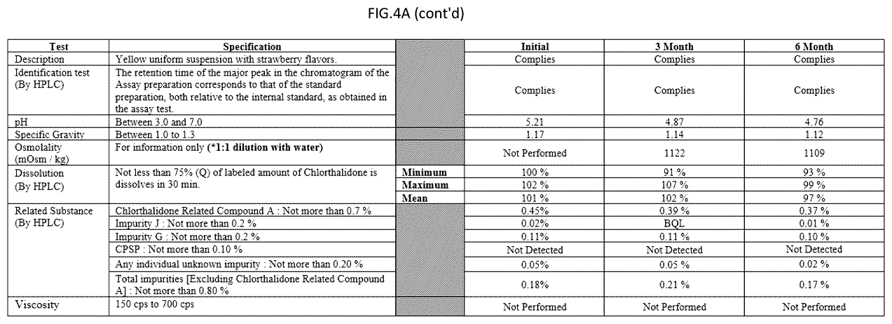

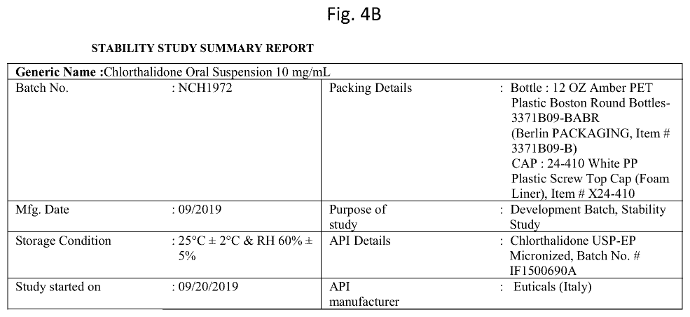

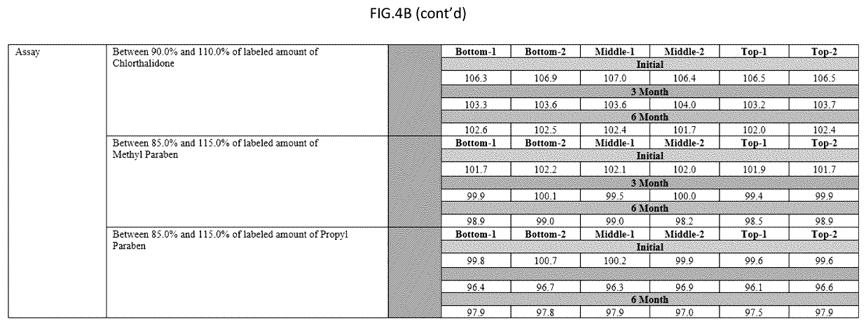

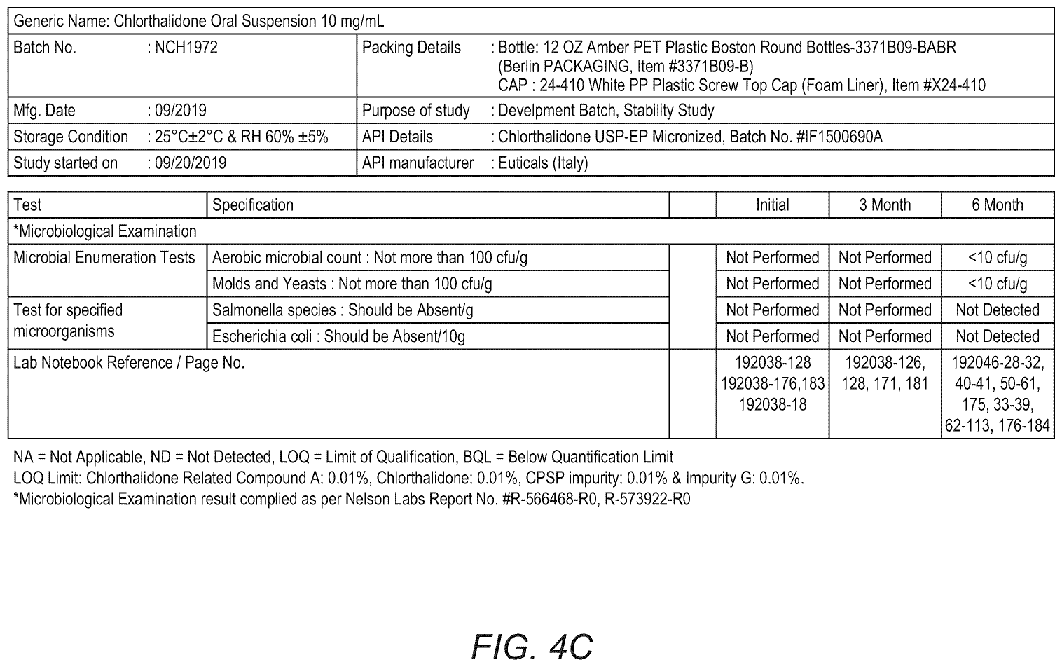

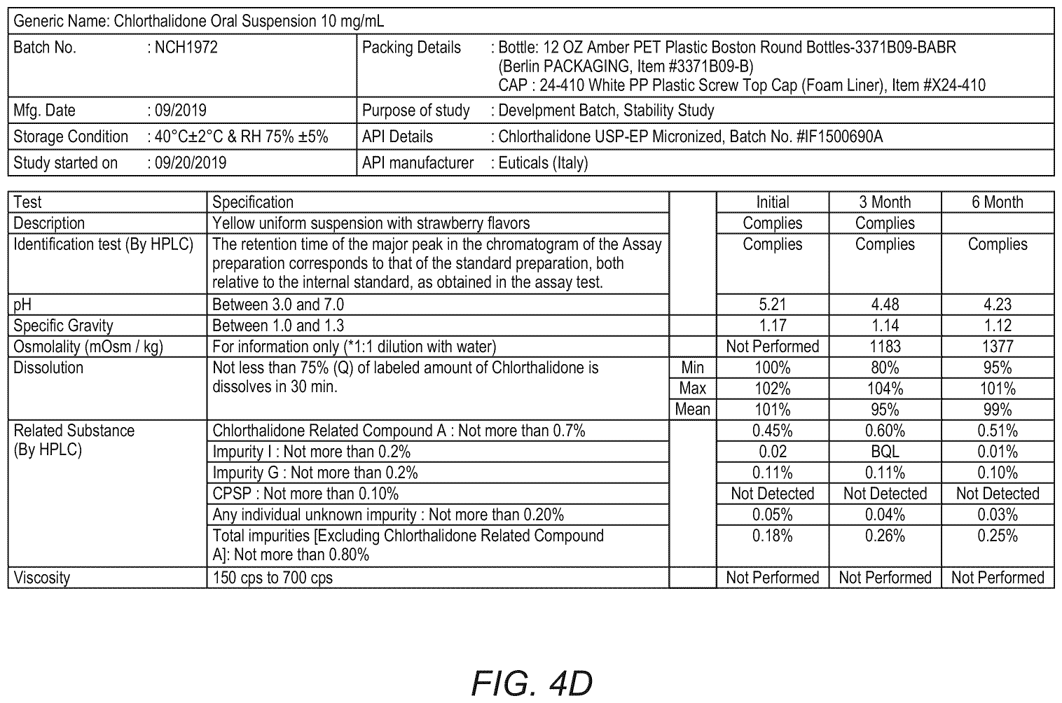

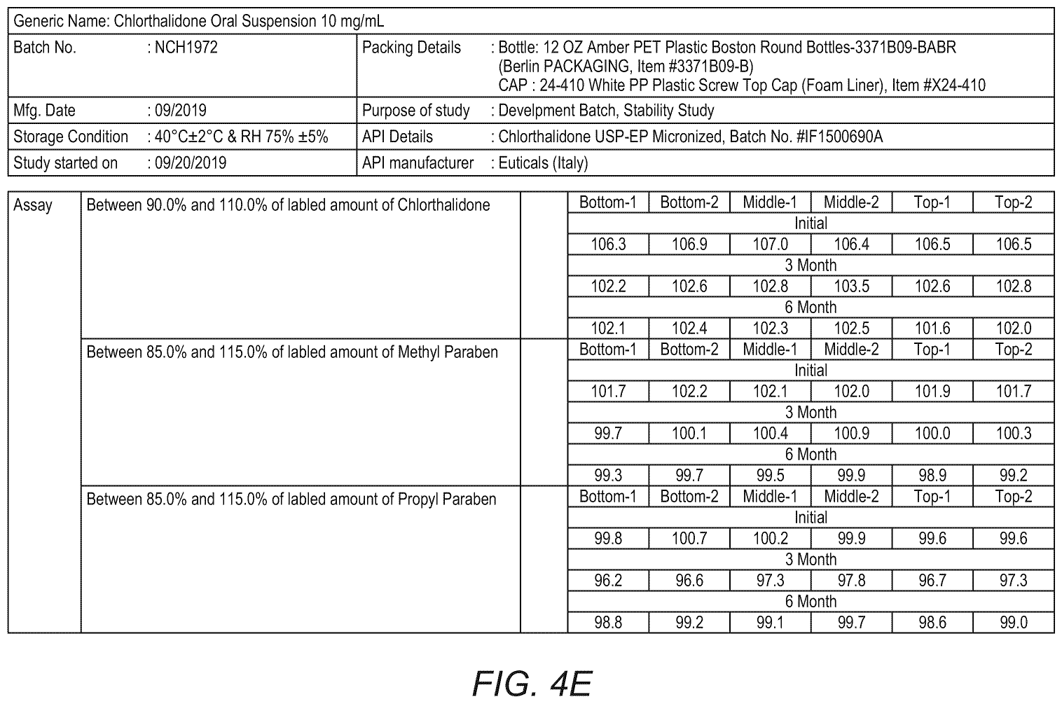

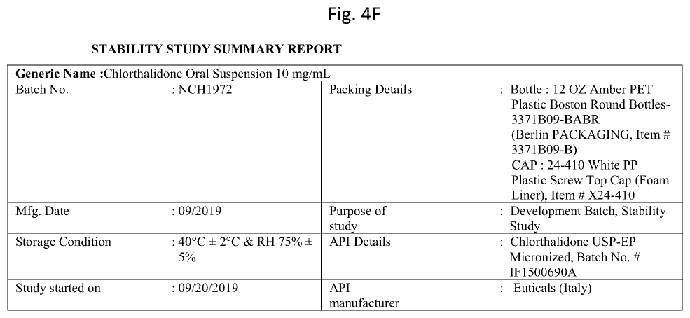

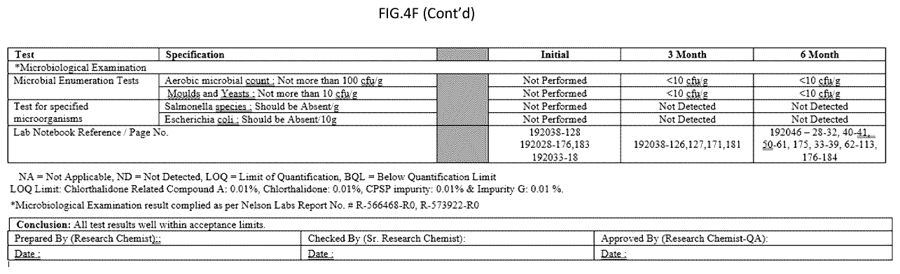

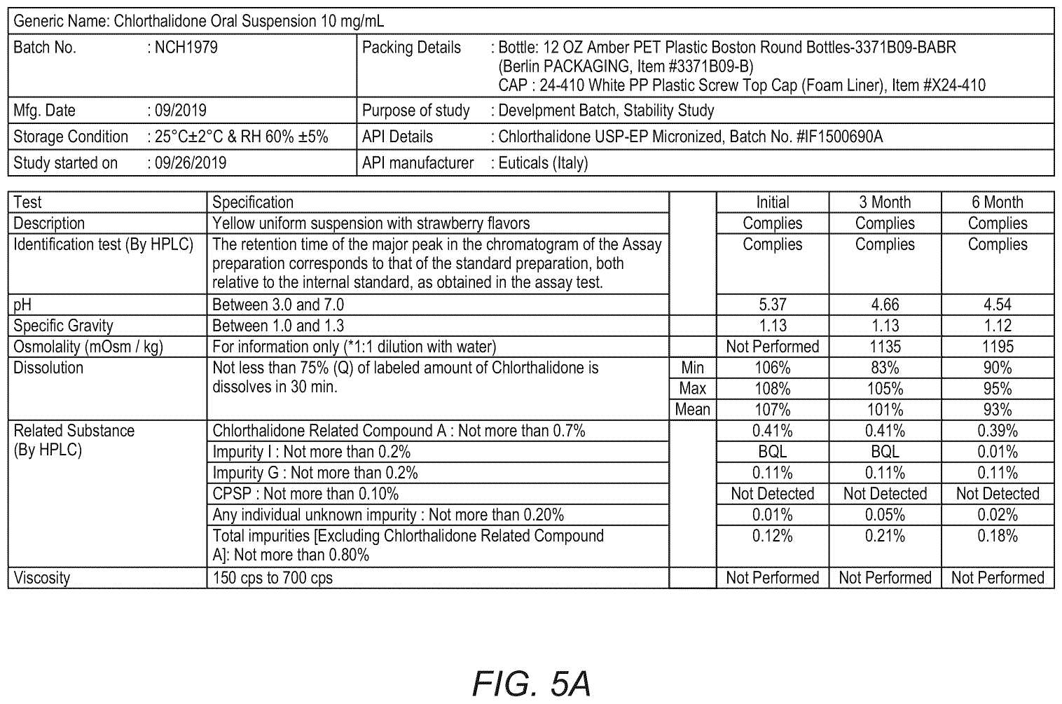

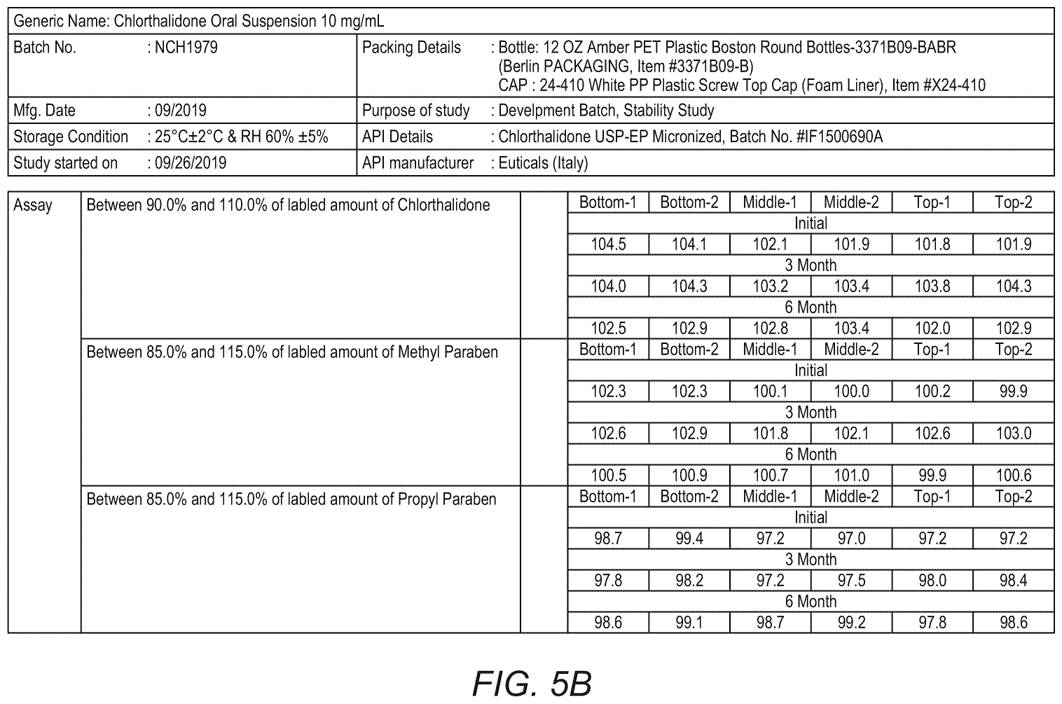

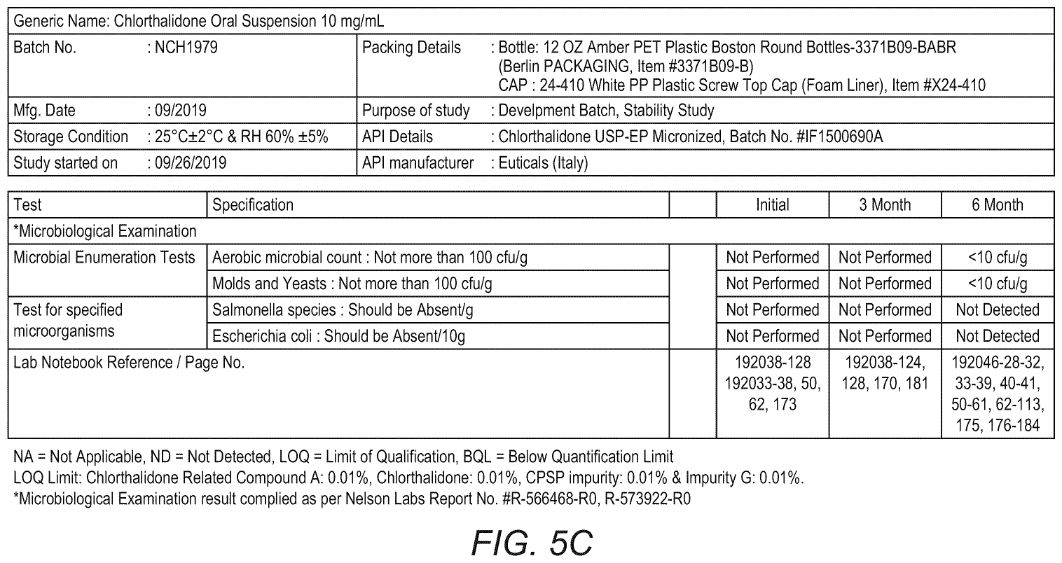

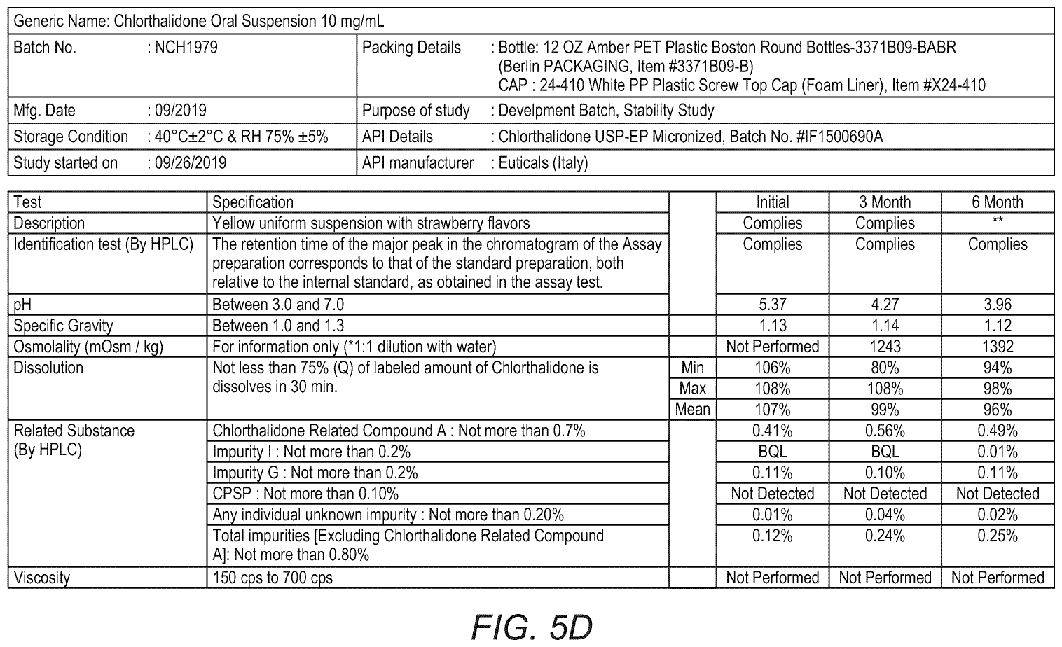

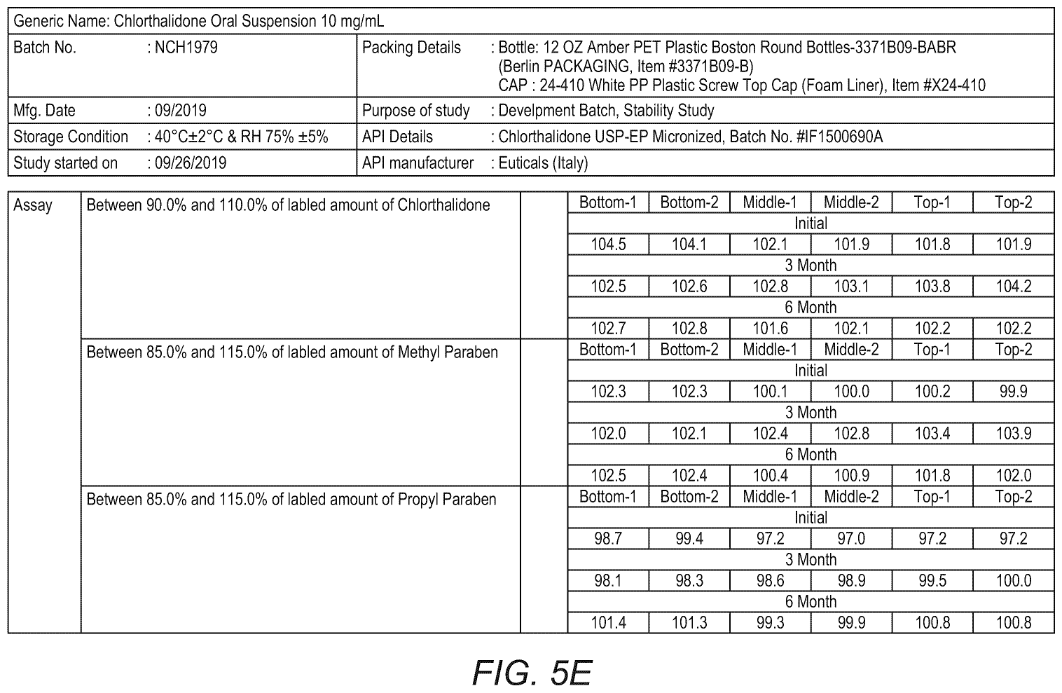

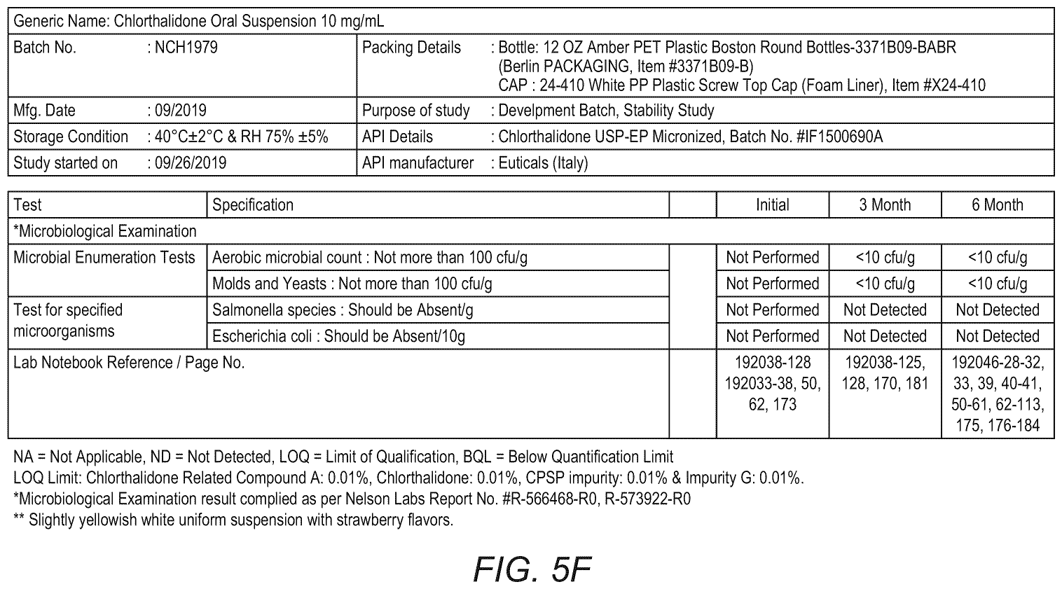

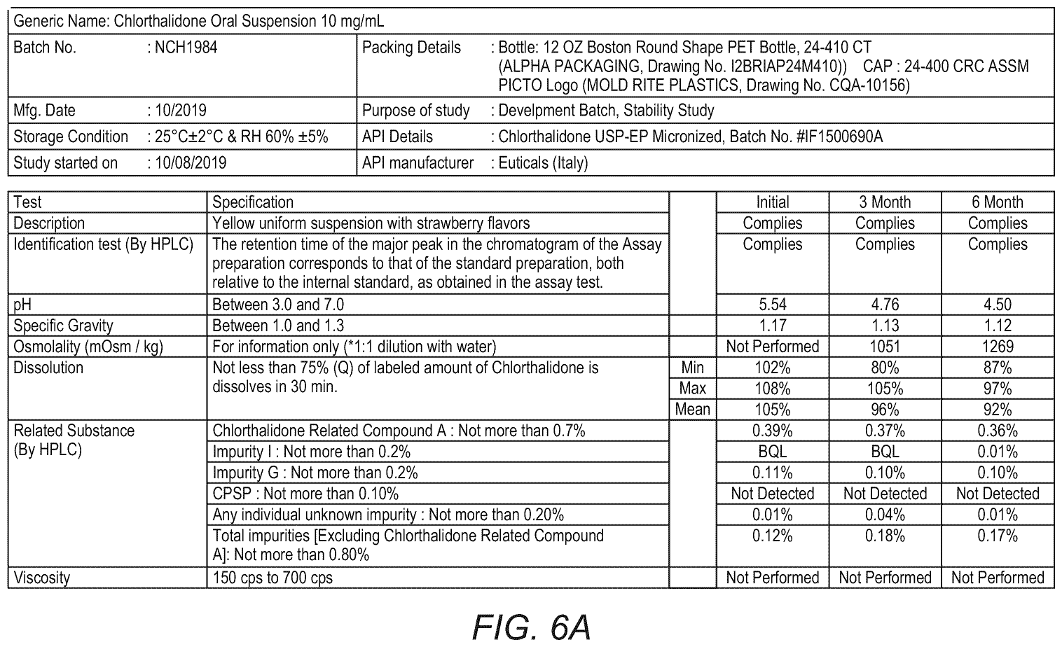

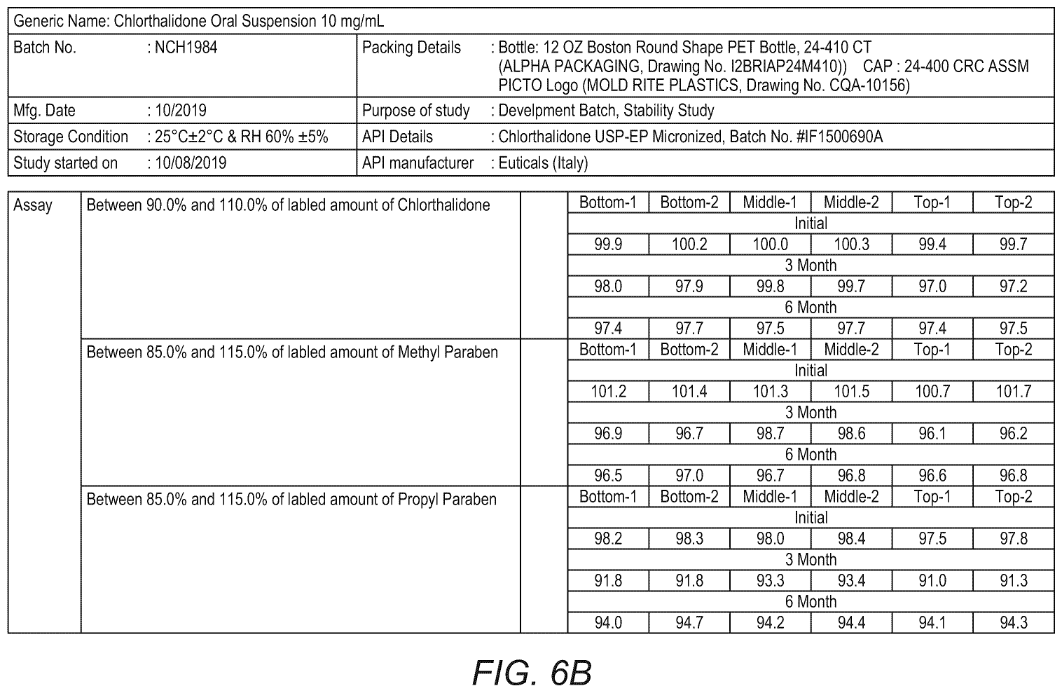

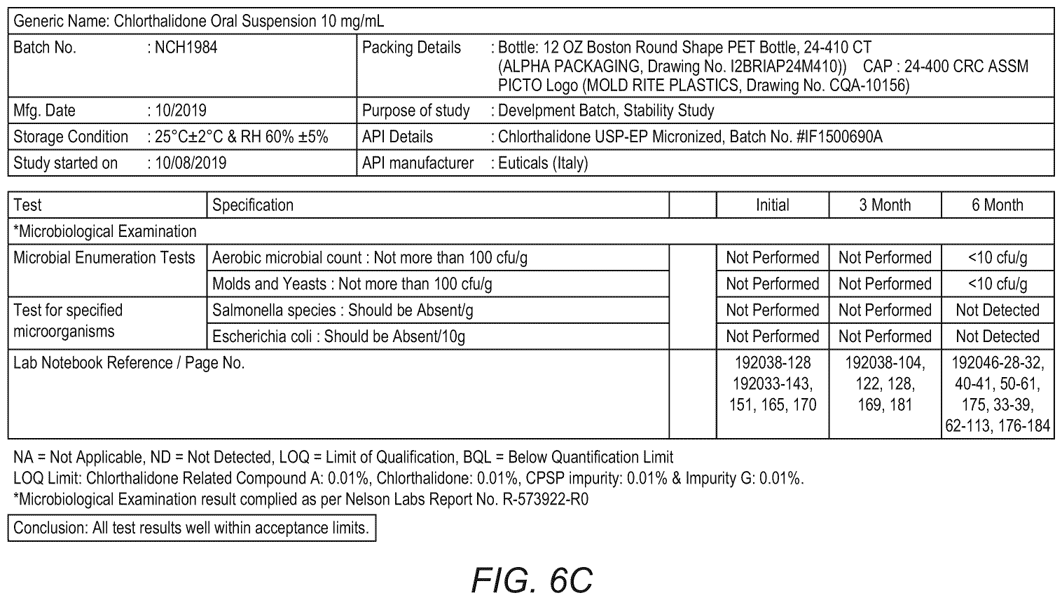

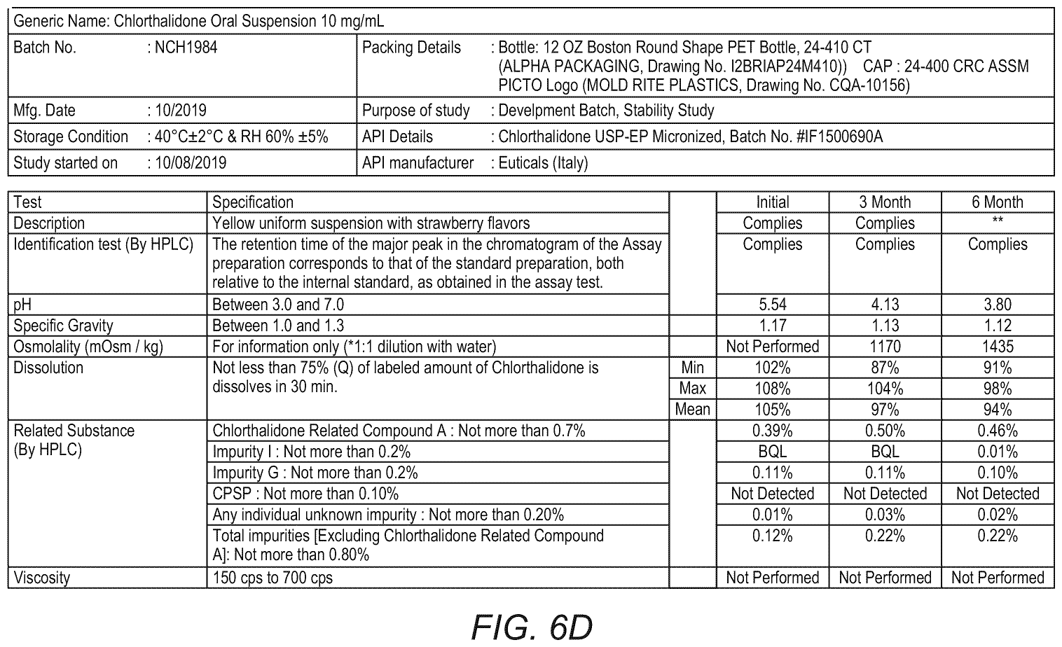

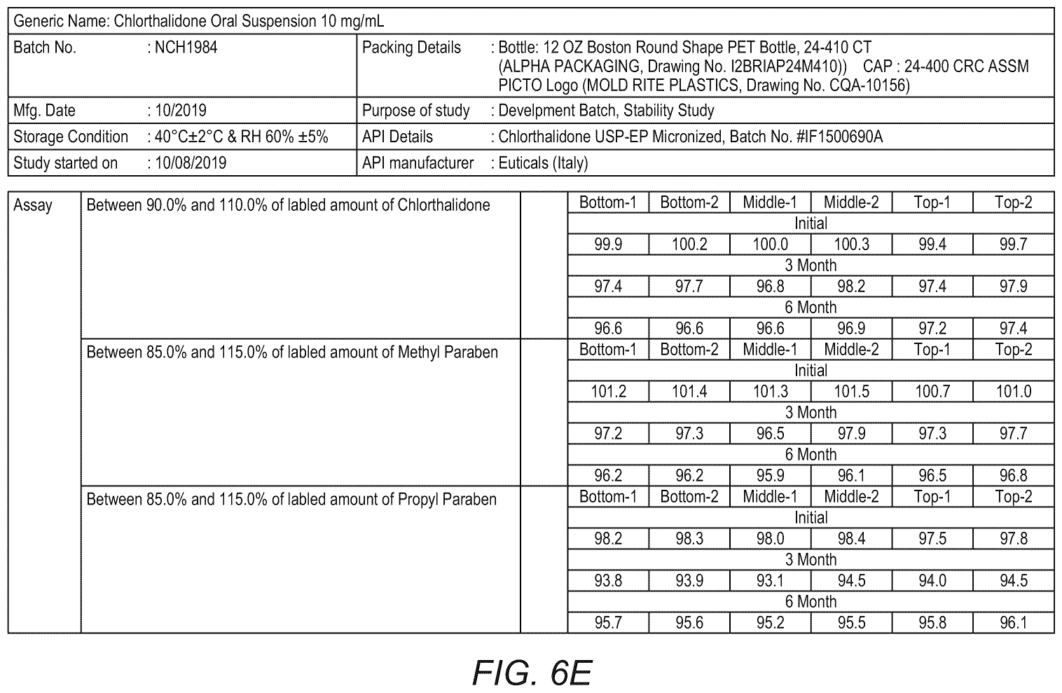

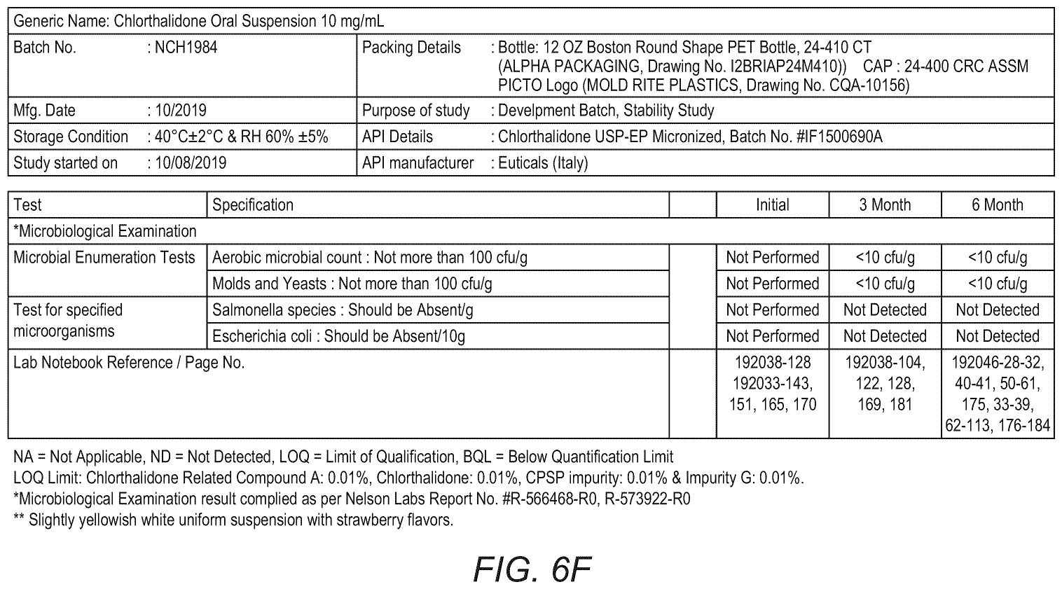

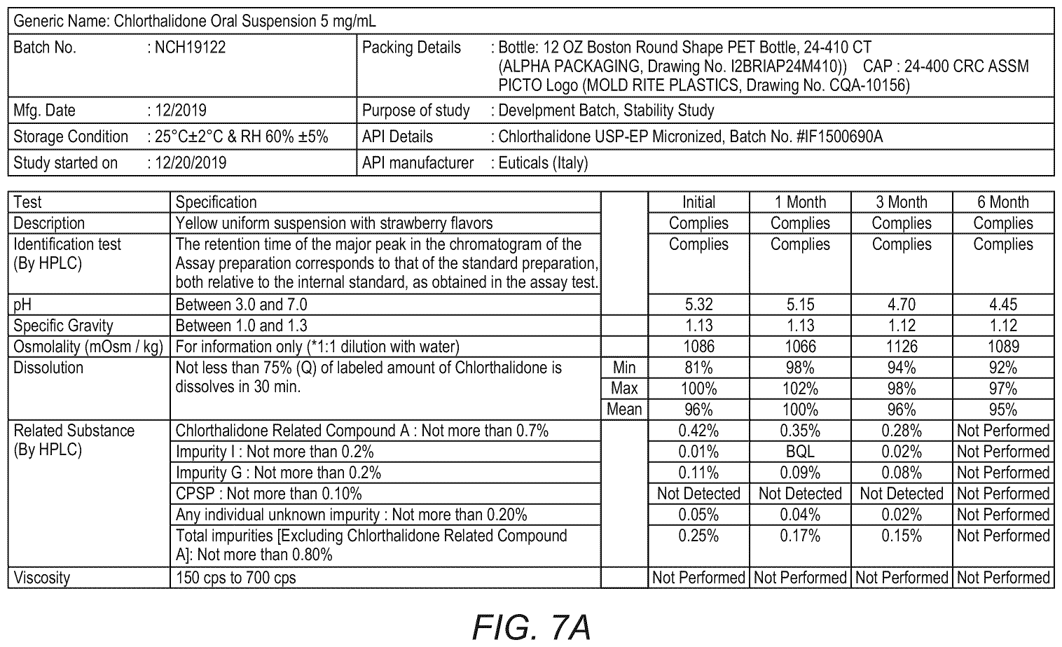

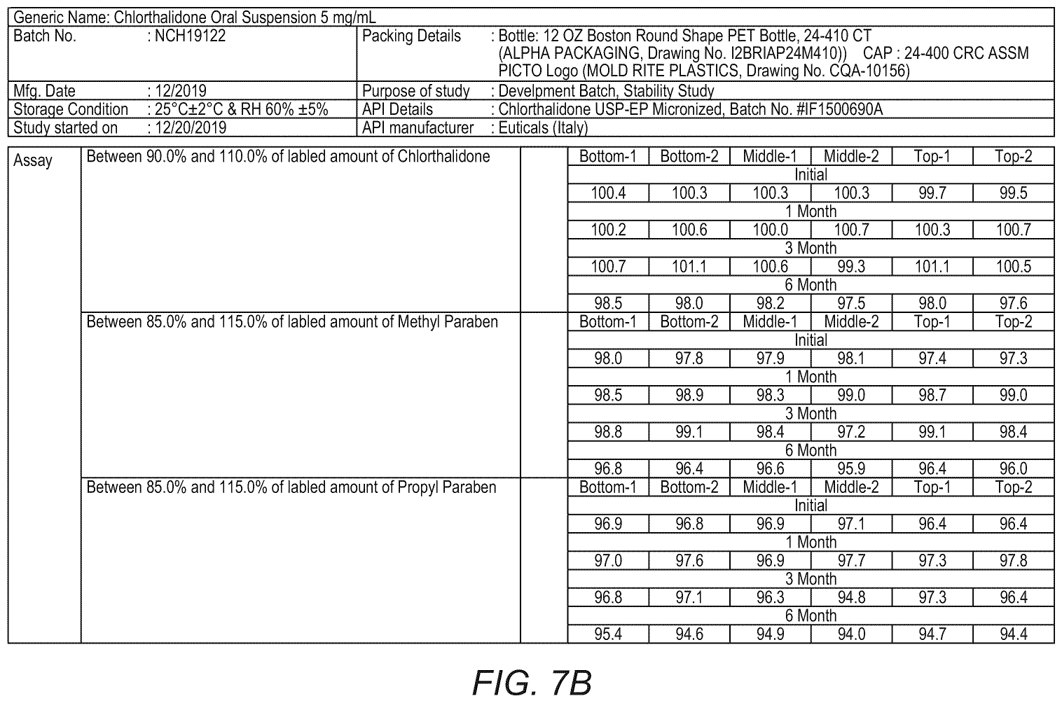

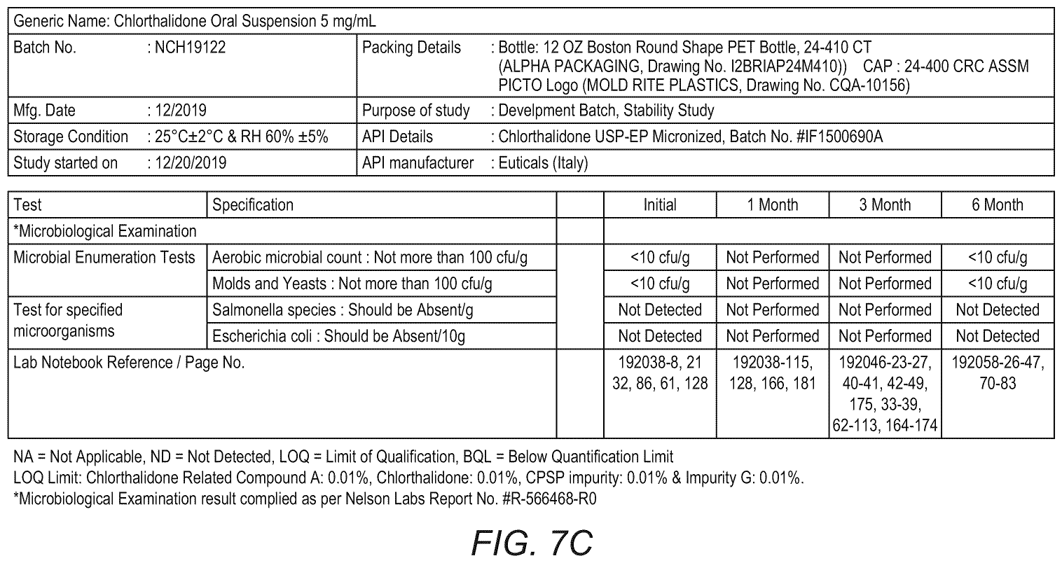

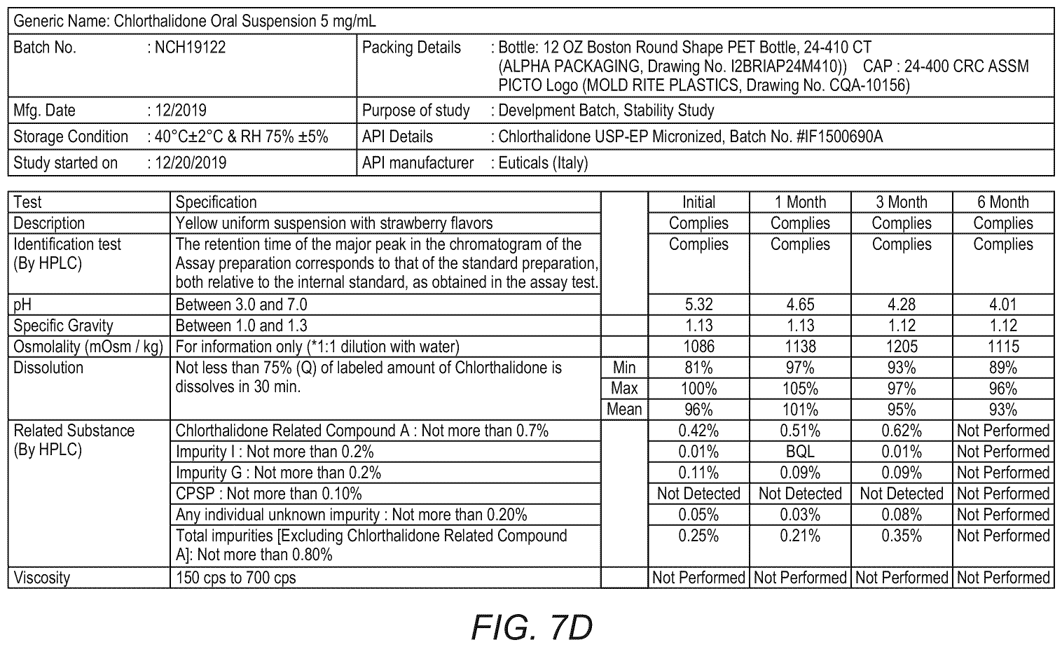

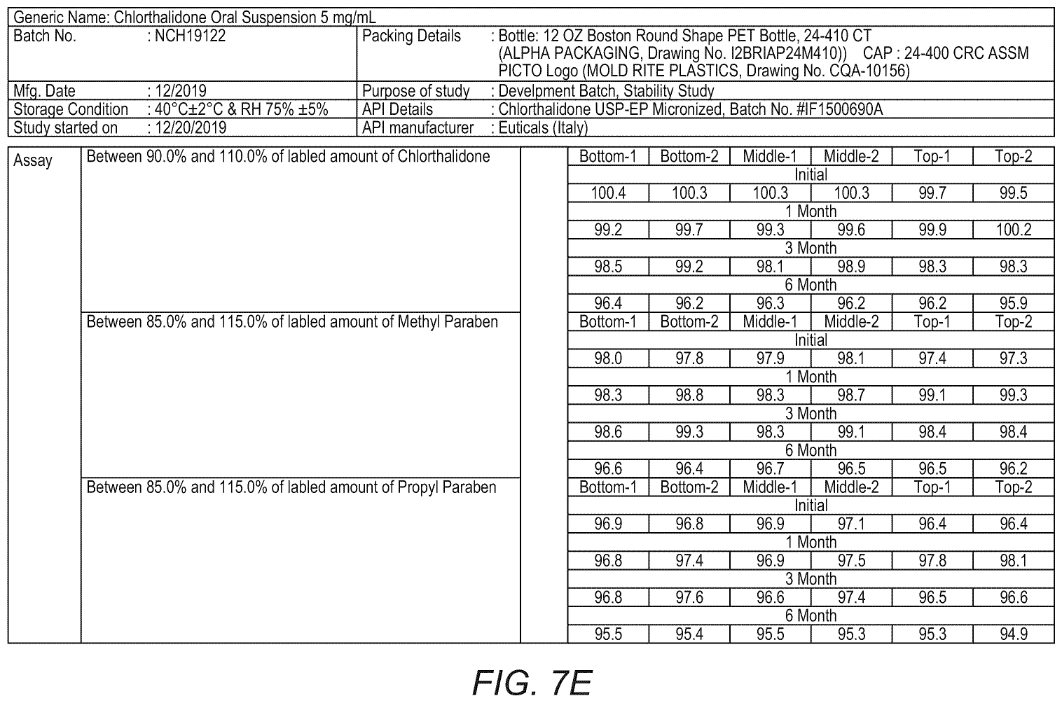

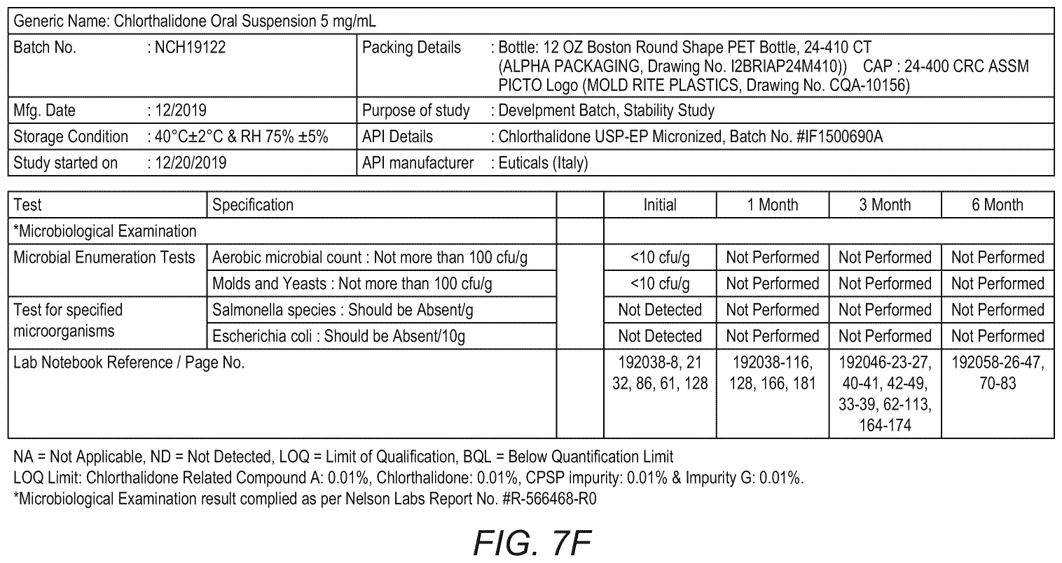

With reference to FIGS. 4A-4F, 5A-5F, 6A-6F, and 7A-7F, stability and settling analysis was carried out at 25° C. and 40° C. as indicated for each of the exemplary chlorthalidone batches of NCH1972, NCH1979, NCH1984, and NC19122.

With reference to Table 6, the batch No. CHLL1009 was prepared with the following composition:

TABLE 6
Composition
Batch No. CHLL1009

| Sr. | | | Quantity |
|---|---|---|---|
| No. | Ingredients | Specification | (mg/mL) |
| 1 | Chlorthalidone | USP | 5.00 |
| 2 | Methyl paraben | NF | 1.80 |
| 3 | Propyl paraben | NF | 0.20 |
| 4 | Sodium Dihydrogen | USP | 1.37 |
| | Phosphate Dihyrate | | |
| 5 | Disodium Hydrogen | USP | 0.22 |
| | Phosphate Dihyrate | | |
| 6 | Sucralose Powder | NF | 0.50 |
| 7 | 30% Simethicone | USP | 5.00 |
| | Emulsion | | |
| 8 | Glycerin | USP | 100.00 |
| 9 | Xanthan Gum | NF | 5.00 |
| 10 | Purified Water | USP | Q.S. to 1.00 mL |

With reference to Table 7 below, the composition of Table 6 was prepared using the following bulk manufacturing steps:

TABLE 7
Bulk manufacturing steps

| 1. | Bulk Manufacturing |
|---|---|
| 1.1 | Tare weight of SS manufacturing vessel (S1) (kg): |
| | Add 80 L Purified water (80% of total batch size) |
| | in SS manufacturing vessel (S1). |
| 1.2 | Increase the temperature of the solution of |
| | Step 1.1 to 75-80° C. and maintain it. |

| | | |
|---|---|---|
| Stirring speed | 200-500 | RPM |
| Temperature | 75-80° | C. |

| | |
|---|---|
| 1.3 | Add dispensed quantity of Methyl paraben in SS |
| | manufacturing vessel (S1) Step 1.2 and stir well |
| | until clear solution is obtained. |

| | | |
|---|---|---|
| Stirring speed | 200-500 | RPM |
| Mixing Time | 10 | min |
| Temperature | 75-80° | C. |

| | |
|---|---|
| 1.4 | Add dispensed quantity of Propyl paraben in SS |
| | manufacturing vessel (S1) Step 1.3 and stir well |
| | until clear solution is obtained. |

| | | |
|---|---|---|
| Stirring speed | 200-500 | RPM |
| Mixing Time | 10 | min |
| Temperature | 75-80° | C. |

| | |
|---|---|
| 1.5 | Cool the solution of Step 1.4, to 20-30° C. |
| | under stirring. |
| | Add purified water to adjust water loss observed |
| | during Step 1.2 to Step 1.4. |

| | | |
|---|---|---|
| Stirring speed | 200-500 | RPM |
| Mixing Time | 10 | min |

| | |
|---|---|
| 1.6 | Add dispensed quantity of Sodium Dihydrogen |
| | Phosphate Dihydrate in SS manufacturing vessel |
| | (S1) Step 1.5 and stir well. |

| | | |
|---|---|---|
| Stirring speed | 200-500 | RPM |
| Mixing Time | 10 | min |

| | |
|---|---|
| 1.7 | Add dispensed quantity of Disodium Hydrogen |
| | Phosphate Dihydrate in SS manufacturing |
| | vessel (S1) Step 1.6 and stir well. |

| | | |
|---|---|---|
| Stirring speed | 200-500 | RPM |
| Mixing Time | 10 | min |

| | |
|---|---|
| 1.8 | Add dispensed quantity of sucralose in SS |
| | manufacturing vessel (S1) |
| | Step 1.7 and stir well. |

| | | |
|---|---|---|
| Stirring speed | 200-500 | RPM |
| Mixing Time | 10 | min |

| | |
|---|---|
| 1.9 | Add dispensed quantity of 30% Simethicone |
| | Emulsion in SS manufacturing |
| | vessel (S1) Step 1.8 and stir well. |

| | | |
|---|---|---|
| Stirring speed | 200-500 | RPM |
| Mixing Time | 10 | min |

| | |
|---|---|
| 1.10 | Tare weight of SS manufacturing vessel (S2) (kg): |
| | Add dispensed quantity of Glycerin in SS |
| | manufacturing vessel (S2). |
| 1.11 | Add dispensed quantity of Chlorthalidone to |
| | SS Manufacturing vessel (S2) |
| | Step 1.10 and stir well. |

| | | |
|---|---|---|
| Stirring speed | 300-500 | RPM |
| Mixing Time | 5 | min |

| | |
|---|---|
| 1.12 | Add dispensed quantity of Xanthan gum to SS |
| | Manufacturing vessel (S2) |
| | Step 1.11 and stir well. |

| | | |
|---|---|---|
| Stirring speed | 300-500 | RPM |
| Mixing Time | 5 | min |

| | |
|---|---|
| 1.13 | Add content of SS Manufacturing vessel (S2) |
| | Step 1.12 to SS Manufacturing vessel (S1) |
| | Step 1.9 and stir well. |

| | | |
|---|---|---|
| Stirring speed | 300-500 | RPM |
| Mixing Time | 60 | min |

| | |
|---|---|
| 1.14 | Add remaining quantity of Purified water in |
| | SS manufacturing vessel (S1) to make up the volume |
| | and mix until uniform suspension is obtained. |

| | | |
|---|---|---|
| Stirring speed | 300-500 | RPM |
| Mixing Time | NLT 15 | min |

| | |
|---|---|
| 1.15 | Connect the SS manufacturing vessel (S1) Step 1.14 |
| | to inline homogenizer to homogenize the suspension. |

| | | |
|---|---|---|
| Homogenization speed | 5000 ± 200 | RPM |
| Homogenization time | 30 | min |

With reference to Table 8 below, stability data for CHLL1009 batch is as follows:

TABLE 8

| | Stability data | | | | | | | | | | | | | | |
|---|---|---|---|---|---|---|---|---|---|---|---|---|---|---|---|
| Sr | | Limit | RS-Mylan | | | | | | | | | | | | |
| 1 | RLD/ | — | 50 mg | Develop- | | Develop- | | Develop- | | Develop- | | Develop- | | Develop- | |
| | Develop- | | Tablet | ment | | ment | | ment | | ment | | ment | | ment | |
| | ment | | | | | | | | | | | | | | |
| 2 | Batch No. | — | 2008545 | CHLL1009 | | CHLL1009 | | CHLL1009 | | CHLL1009 | | CHLL1009 | | CHLL1009 | |
| 4 | T0/ | — | T0 | T0 | | 40° C./25% | | 40° C./25% | | 40° C./25% | | 25° C./40% | | 25° C./40% | |
| | Stability | | | | | −1 | Month | −3 | Months | −6 | Months | −3 | Months | −6 | Months |
| | Time Point | | | | | | | | | | | | | | |
| 5 | Pack | — | HDPE | 330 | ml | 330 | mL | 330 | ml | 330 | ml | 330 | ml | 330 | ml |
| | | | Bottle | White PET | | White PET | | White PET | | White PET | | White PET | | White PET | |
| 6 | API source | — | — | AMRI | | AMRI | | AMRI | | AMRI | | AMRI | | AMRI | |
| | Lot No. | | — | IF1902346A | | IF1902346A | | IF1902346A | | IF1902346A | | IF1902346A | | IF1902346A | |
| | API PSD | | — | D(100) | | D(100) | | D(100) | | D(100) | | D(100) | | D(100) | |
| | | | | 60 | μm | 60 | μm | 60 | μm | 60 | μm | 60 | μm | 60 | μm |
| 7 | Appearance | — | Light green, | Off white | | Off white | | Off white | | Off white | | Off white | | Off white | |
| | | | round, scored | suspension | | suspension | | suspension | | suspension | | suspension | | suspension | |
| | | | tablets de- | | | | | | | | | | | | |
| | | | bossed with | | | | | | | | | | | | |
| | | | M to the left | | | | | | | | | | | | |
| | | | of the score | | | | | | | | | | | | |
| | | | and 75 to | | | | | | | | | | | | |
| | | | the right of | | | | | | | | | | | | |
| | | | the score on | | | | | | | | | | | | |
| | | | one side of | | | | | | | | | | | | |
| | | | the tablet and | | | | | | | | | | | | |
| | | | blank on the | | | | | | | | | | | | |
| | | | other side. | | | | | | | | | | | | |
| 8 | Viscosity | To be | NA | 157.5 | cps | 155.5 | cps | 151.5 | cps | 141.5 | cps | 155.5 | cps | 149.0 | cps |
| | | recorded | | | | | | | | | | | | | |
| 9 | Assay of | 90 to | 99.6% | 101.4% | | 101.5% | | 101.7% | | 102.6% | | 101.5% | | 101.3% | |
| | Chlortha- | 110% | | | | | | | | | | | | | |
| | lidone | | | | | | | | | | | | | | |
| 10.1 | Content of | 80 to | NA | 97.1% | | 97.0% | | 96.7% | | 97.0% | | 97.5% | | 97.2% | |
| | Methyl | 110% | | | | | | | | | | | | | |
| 10.2 | Content of | 80 to | NA | 97.5% | | 97.6% | | 97.0% | | 98.9% | | 97.7% | | 99.4% | |
| | Propyl | 110% | | | | | | | | | | | | | |
| | paraben | | | | | | | | | | | | | | |
| 11 | pH | To be | — | 5.9 | | 5.8 | | 5.5 | | 5.1 | | 5.8 | | 5.7 | |
| | | recorded | | | | | | | | | | | | | |
| 12 | Specific | To be | NA | 1.01 | | 1.01 | | 1.01 | | 1.02 | | 1.01 | | 1.01 | |
| | Gravity | recorded | | | | | | | | | | | | | |
| | (gm/mL) | | | | | | | | | | | | | | |
| 13 | Zeta | To be | NA | −22.6 | | −27.9 | | −28.4 | | −22.2 | | −24.8 | | −19.8 | |
| | Potential | recorded | | | | | | | | | | | | | |
| | (mV) | | | | | | | | | | | | | | |
| 14 | Particle | | | | | | | | | | | | | | |
| | Size | | | | | | | | | | | | | | |
| | Distribution | | | | | | | | | | | | | | |
| | D(10) | To be | NA | 2.0 | μm | 2.1 | μm | 2.0 | μm | 2.0 | μm | 2.0 | μm | 2.0 | μm |
| | | recorded | | | | | | | | | | | | | |
| | D(50) | To be | NA | 6.4 | μm | 7.0 | μm | 7.0 | μm | 6.3 | μm | 6.8 | μm | 6.4 | μm |
| | | recorded | | | | | | | | | | | | | |
| | D(90) | To be | NA | 19.5 | μm | 20.6 | μm | 23.2 | μm | 17.5 | μm | 22.1 | μm | 20.0 | μm |
| | | recorded | | | | | | | | | | | | | |

| | | | | | | | | | |
|---|---|---|---|---|---|---|---|---|---|
| 15 | Related | | | | | | | | |
| | Substances | | | | | | | | |
| 15.1 | Chlortha- | NMT | 0.29% | 0.39% | 0.39% | 0.46% | 0.44% | 0.41% | 0.38% |
| | lidone | 1.0% | | | | | | | |
| | Related | | | | | | | | |
| 15.2 | Impurity-J | NMT | ND | ND | ND | ND | ND | ND | ND |
| | | 0.2% | | | | | | | |
| 15.3 | Impurity-G | NMT | 0.17% | 0.13% | 0.13% | 0.14% | 0.13% | 0.14% | 0.13% |
| | | 0.2% | | | | | | | |
| 15.4 | Impurity-E | NMT | ND | ND | ND | ND | ND | ND | ND |
| | (CPSP) | 0.1% | | | | | | | |
| 15.5 | Single Max | NMT | 0.05% | 0.08% | 0.07% | 0.10% | 0.19% | 0.07% | 0.09% |
| | unspecified | 0.2% | | | | | | | |
| 15.6 | Total | NMT | 0.72% | 0.79% | 0.81% | 1.06% | 1.18% | 0.88% | 0.82% |
| | impurities | 1.2% | | | | | | | |

Finally, with regard to Table 9 below, dissolution data in various media is presented below:

TABLE 9
Multimedia dissolution data

| Chlorthalidone Suspension | | | | | | | | | | | | |
|---|---|---|---|---|---|---|---|---|---|---|---|---|
| 5 mg/mL-Multimedia Dissolution | | | | | | | | | | | | |
| | 75 RPM | | | | | | | | | | | F2 with |
| RPM | API Lot No/ | | | | | | | | | | F2 with | Nivagen |
| Batch No. | Particle Size | Media | | 5 min | 10 min | 15 min | 30 min | 45 min | 60 min | 75 min | RS-Mylan | Bio Batch |
| CHBU03 | Nivagen | OGD | Drug | 57 | 69 | 76 | 87 | 92 | 95 | — | 50 | NA |
| Bio Batch | Bio Batch | (Purified | Release | | | | | | | | | |
| | | Water) | | | | | | | | | | |
| | | 0.1 N HCL | Drug | 51 | 66 | 76 | 91 | 97 | 102 | 104 | 48 | NA |
| | | | Release | | | | | | | | | |
| | | pH 4.5 | Drug | 52 | 65 | 74 | 86 | 91 | 93 | 96 | 56 | NA |
| | | Acetate | Release | | | | | | | | | |
| | | Buffer | | | | | | | | | | |
| | | pH 6.8 | Drug | 52 | 65 | 72 | 83 | 88 | 91 | 93 | 47 | NA |
| | | Phosphate | Release | | | | | | | | | |
| | | Buffer | | | | | | | | | | |
| 2009364 | Mylan-RS | OGD | Drug | 40.9 | 55.7 | 64.5 | 78.1 | 84.7 | 88.6 | 91.1 | NA | NA |
| RS 50 mg | | (Purified | Release | | | | | | | | | |
| | | Water) | | | | | | | | | | |
| | | 0.1 N HCL | Drug | 32.2 | 48.3 | 57.9 | 73.1 | 80.4 | 84.8 | 87.7 | NA | NA |
| | | | Release | | | | | | | | | |
| | | pH 4.5 | Drug | 34.6 | 49.6 | 58.6 | 73.1 | 80.3 | 84.8 | 87.8 | NA | NA |
| | | Acetate | Release | | | | | | | | | |
| | | Buffer | | | | | | | | | | |
| | | pH 6.8 | Drug | 35.3 | 49.5 | 58.1 | 72.2 | 79.4 | 83.9 | 87.0 | NA | NA |
| | | Phosphate | Release | | | | | | | | | |
| | | Buffer | | | | | | | | | | |
| CHLL1009 | AMRI | OGD | Drug | 78.8 | 91.3 | 95.4 | 97.5 | 97.6 | 97.5 | 97.6 | 29 | 36 |
| | IF1902346A | (Purified | Release | | | | | | | | | |
| | D100 = | Water) | | | | | | | | | | |
| | 60 μm | 0.1 N HCL | Drug | 86.8 | 94.6 | 97.1 | 99.0 | 99.3 | 99.3 | 99.4 | 23 | 30 |
| | (D90 = | | Release | | | | | | | | | |
| | 14 μm) | pH 4.5 | Drug | 78.9 | 87.6 | 94.2 | 98.9 | 98.8 | 98.8 | 100.8 | 27 | 34 |
| | | Acetate | Release | | | | | | | | | |
| | | Buffer | | | | | | | | | | |
| | | pH 6.8 | Drug | 55.1 | 69.5 | 74.9 | 90.1 | 94.9 | 97.6 | 101.3 | 38 | 64 |
| | | Phosphate | Release | | | | | | | | | |
| | | Buffer | | | | | | | | | | |

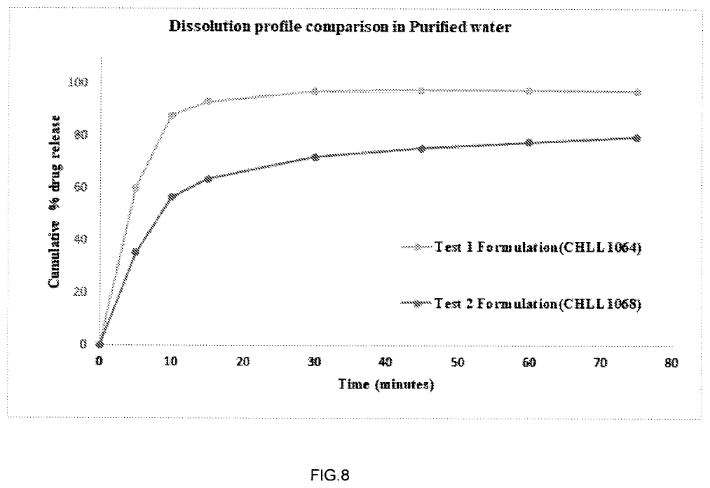

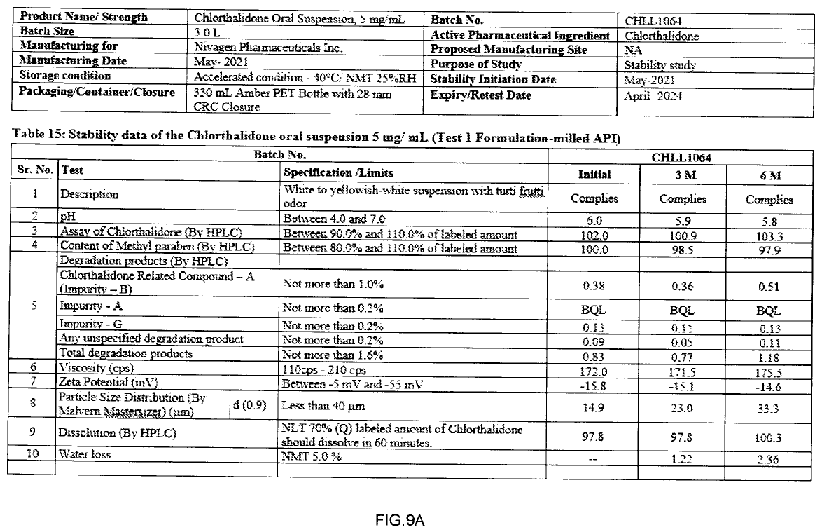

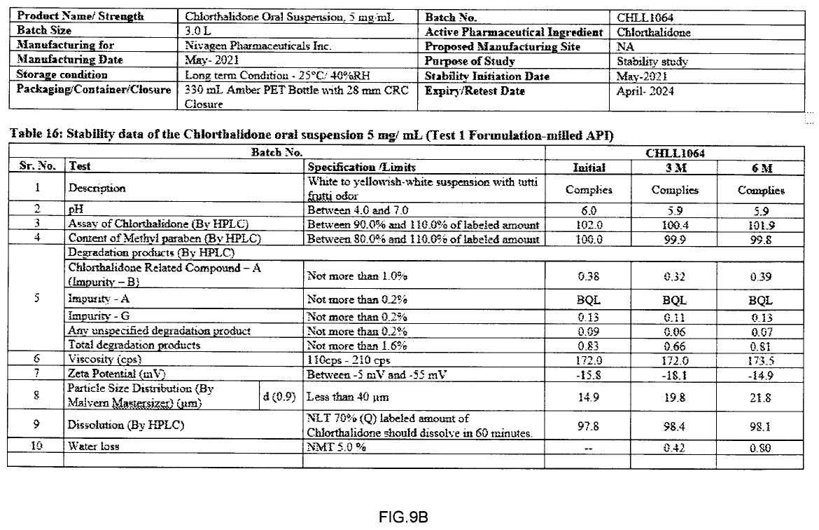

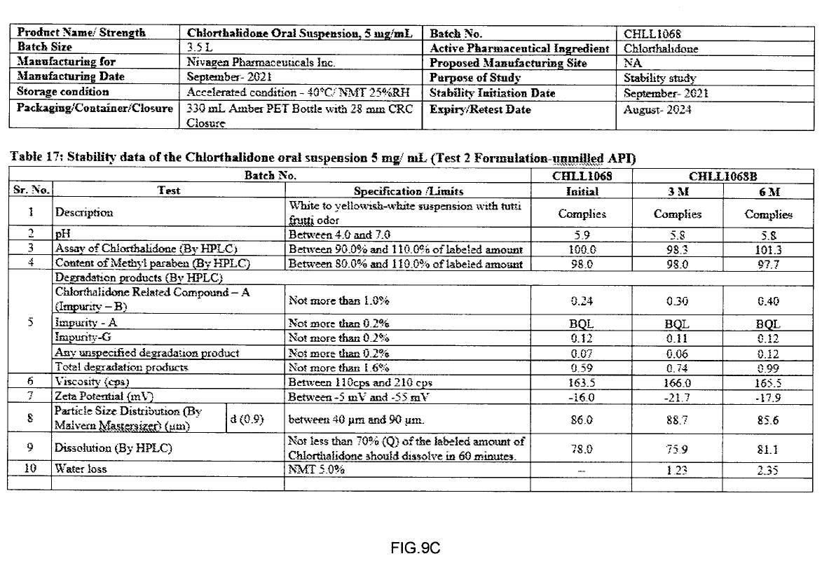

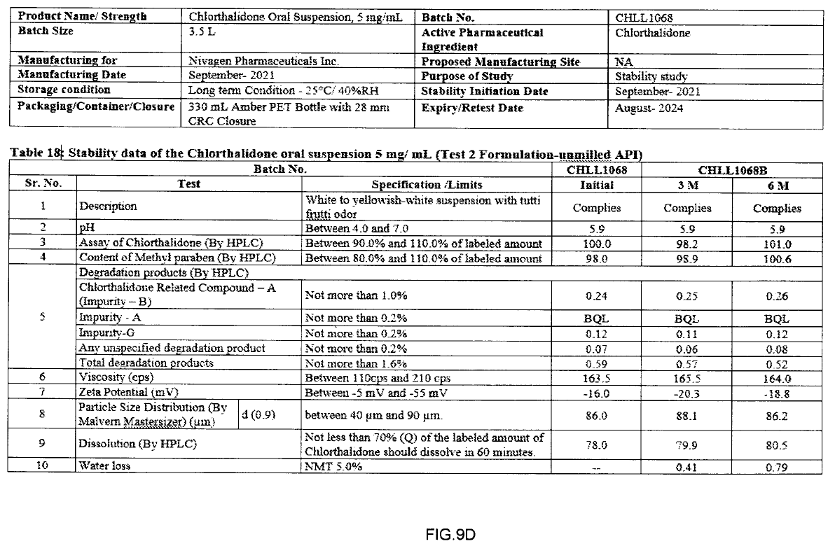

Still further dissolution data are provided in Table 10 below, which are also illustrated as a graph in FIG. 8.

TABLE 10
Comparative Dissolution profile of Test 1 Formulation-milled
API (B. No. CHLL1064) and Test 2 Formulation-unmilled API
(B. No. CHLL1068) in Release Media (Purified water).

| | Test 1 Formulation- | Test 2 Formulation- |
|---|---|---|
| Time points | milled API | unmilled API |
| (min) | (B. No. CHLL1064) | (B. No. CHLL1068) |
| 0 | 0 | 0 |
| 5 | 60.3 | 35.5 |
| 10 | 88.0 | 56.8 |
| 15 | 93.4 | 63.7 |
| 30 | 97.4 | 72.2 |
| 45 | 97.9 | 75.6 |
| 60 | 97.8 | 78.0 |
| 75 | 97.5 | 80.0 |

Therefore, in view of the above, the inventors also contemplate an exemplary formulation of a suspension that has the exemplary product specifications listed in Table 11, and an exemplary composition for such suspension is show in Table 12. Exemplary methods to produce such formulations from milled or un-milled chlorthalidone are show in tables 13 and 14, respectively.

TABLE 11
Exemplary product specification.

| Sr. | | | | | | |
|---|---|---|---|---|---|---|
| No. | Test | Release Specification | | Shelf-Life Specification | | Reference |
| 1. | Description | White to yellowish-white | | White to yellowish-white | | In-House |
| | | suspension. | | suspension. | | |
| 2. | pH | Between 5.0 and 7.0 | | Between 4.0 and 7.0 | | USP <791> |
| 3. | Assay of | Not less than 90.0% and | | Not less than 90.0% and | | In-House |
| | Chlorthalidone | Not more than 110.0% | | Not more than 110.0% | | |
| | (By HPLC) | | | | | |
| | (Each mL contains: | | | | | |
| | Chlorthalidone | | | | | |
| | USP_5.0 mg) | | | | | |
| 4. | Content of Methyl | Not less than 90.0% and | | Not less than 80.0% and | | In-House |
| | paraben (By HPLC) | Not more than 110.0% | | Not more than 110.0% | | |
| | (Each mL contains: | | | | | |
| | Methyl Paraben_1.0 | | | | | |
| | mg) | | | | | |
| 5. | Degradation products (By HPLC) | | | | | In-House |
| | Chlorthalidone related | Not more than 1.0% | | Not more than 1.0% | | |
| | compound-A (Impurity | | | | | |
| | B) | | | | | |
| | Impurity A | Not more than 0.2% | | Not more than 0.2% | | |
| | Impurity G | Not more than 0.2% | | Not more than 0.2% | | |
| | Any unspecified | Not more than 0.2% | | Not more than 0.2% | | |
| | degradation product | | | | | |
| | Total degradation | Not more than 1.6% | | Not more than 1.6% | | |
| | products | | | | | |
| 6. | Viscosity | 110 cps-210 cps | | 110 cps-210 cps | | In-House |
| 7. | Zeta Potential | Between −5 mV and −55 mV | | Between −5 mV and −55 mV | | In-House |
| 8. | Particle size distribution* (By Malvern Mastersizer) | | | | | In-House |
| | Formulation-1 | d(0.9) | Less than 40 μm | d(0.9) | Less than 40 μm | In-House |
| | (milled API) | | | | | |
| | Formulation-2 | d(0.9) | Less than 90 μm | d(0.9) | Less than 90 μm | In-House |
| | (unmilled API) | | | | | |

| | | | | |
|---|---|---|---|---|
| 9. | Dissolution | Not less than 70% (Q) of | Not less than 70% (Q) of | In-House |
| | (By HPLC) | the labeled amount of | the labeled amount of | |
| | | Chlorthalidone should | Chlorthalidone should | |
| | | dissolve in 60 minutes. | dissolve in 60 minutes. | |
| 10. | Water loss | Not applicable | Not more than 5% from | In-House |
| | | | its initial value. | |

| Impurity Name | Chemical Name/IUPAC Name |
|---|---|
| Chlorthalidone Related | 2-(4-Chloro-3-Sulfamoylbenzoyl) |
| compound-A (Impurity B) | benzoic acid |
| Impurity A | 2-(4-chloro-3-sulfobenzoyl)benzoic acid |
| Impurity G | (3RS)-3-(3, 4-dichlorophenyl)-3-hydroxy-2,3- |
| | dihydro-1H-isoindol-1-one. |

TABLE 12
Exemplary formulation composition
(Milled API and Un-milled API):

| Sr. no. | Ingredients | mg/mL |
|---|---|---|
| 1 | Chlorthalidone, USP | 5.00 |
| 2 | Methylparaben, NF | 1.00 |
| 3 | Monosodium phosphate monohydrate, USP | 6.05 |
| 4 | Dibasic sodium phosphate anhydrous, USP | 0.87 |
| 5 | Sucralose, NF | 0.50 |
| 6 | Simethicone Emulsion, USP | 5.00 |
| 7 | Glycerin, USP | 100.00 |
| 8 | Xanthan Gum, NF | 5.00 |
| 9 | Tutti frutti flavor | 0.10 |
| 10 | Purified water, USP | Q.S. to 1 mL |

TABLE 13
Exemplary manufacturing procedure (Milled API):

| STEP | MANUFACTURING PROCEDURE |
|---|---|
| No. | Main Process Tank |
| 1. | Add 40% of total batch size of Purified water, USP, |
| | into the Main Process Tank. Start mixing with high |
| | shear mixer. Heat it to 70° C.(75-85° C.) |
| 2. | To the Main Process Tank, add and dissolve the |
| | Methylparaben, NF. |
| | Note: |
| | Maintain the temperature to 70° C. (75-85° C.) |
| | through Step 3. |
| 3. | Mix Step 2 for NLT Thirty (30) Minutes or until a |
| | clear solution is achieved. |
| 4. | Add 40% of total batch size of Purified water, USP, |
| | into the Main Process Tank. |
| | Cool the solution to 25-30° C. |
| 5. | To the Main Process Tank, add and dissolve the |
| | Monosodium phosphate monohydrate, USP. |
| 6. | Mix Step 5 for NLT Ten (10) Minutes and until a |
| | clear solution is achieved. |
| 7. | To the Main Process Tank, add and dissolve the |
| | Dibasic Sodium phosphate anhydrous, USP. |
| 8. | Mix Step 7 for NLT Ten (10) Minutes or until a |
| | clear solution is achieved. |
| 9. | To the Main Process Tank, add and dissolve the |
| | Sucralose, NF. |
| 10. | Mix Step 9 for NLT Ten (10) Minutes or until a |
| | clear solution is achieved. |
| 11. | To the Main Process Tank, add and mix the Simethicone |
| | Emulsion, USP slowly. |
| | Rinse the container using Purified water, USP: 1.0 kg. |
| | Add the rinse to Main Process Tank. |
| 12. | Mix Step 11 for NLT Ten (10) Minutes or until a |
| | uniform dispersion is achieved. |
| 13. | Weigh the Glycerin Natural 99.7%, USP in Separate |
| | SS vessel. |
| 14. | To the SS vessel (Step13), add the Chlorthalidone, USP. |
| | Mix for NLT Ten (10) Minutes or until a uniform |
| | dispersion is achieved. |
| 15. | To the SS vessel (Step14), add the Xanthan gum, |
| | NF slowly. |
| 16. | Transfer the dispersion from Step # 15 into Main |
| | Process Tank (Step # 12) under stirring. |
| | Rinse the SS vessel with Purified Water, USP and add |
| | the rinse to the Main Process Tank under stirring. |
| | Mix for NLT Sixty (60) Minutes until to get uniform |
| | suspension. |
| 17. | To the Main Process Tank, add the Tutti Frutti flavor. |
| | Rinse the SS vessel with Purified Water, USP [1.00 kg] |
| | and add the rinse to the Main Process Tank under stirring. |
| 18. | Add remaining quantity of Purified water to make up the |
| | volume to batch size. |
| 19. | Connect the outlet of the Main Process Tank to the inlet |
| | of the Admix Fast feed unit and outlet of the Admix Fast |
| | feed unit to the inlet of Main Process Tank. Start high shear |
| | mixing in the Main Process Tank. |
| | Pass the suspension through Fast feed Max (FFD-01) with |
| | Dynashear turned OFF for Thirty (30) minutes. |
| | pH (5.0-7.0): |
| | Suspension Description (White to off white |
| | suspension): |
| | Particle size distribution (By Malvern Mastersizer): |
| | [D90: less than 40 μm] |
| 20. | Close the Process Tank with a lid and label the tank |
| | with product name, Lot number, Quantity. |
| 21. | Withdraw approximately 400 grams of the following bulk |
| | pre-filtration samples from the Process Tank |
| | Top, Middle and Bottom samples: Submit to QC for testing. |
| 22. | Hold the batch in the Process Tank for filling/packaging. |

TABLE 14
Exemplary manufacturing procedure (Un-milled API):

| STEP | MANUFACTURING PROCEDURE |
|---|---|
| No. | Intermix Tank |
| 1. | Add 40% of total batch size of Purified water, USP, |
| | into the Intermix Tank. Start mixing |
| | with high shear mixer. Heat it to 80° C.(75-85° C.) |
| 2. | To the Intermix Tank, add and dissolve the Methylparaben, |
| | NF. |
| | Note: |
| | Maintain the temperature to 80° C. (75-85° C.) through |
| | Step 3. |
| 3. | Mix Step 2 for NLT Thirty (30) Minutes or until a clear |
| | solution is achieved. |
| 4. | Cool the solution to 25-30° C. |
| 5. | To the Intermix Tank, add and dissolve the Monosodium |
| | phosphate monohydrate, USP. |
| 6. | Mix Step 5 for NLT Ten (10) Minutes and until a clear |
| | solution is achieved. |
| 7. | To the Intermix Tank, add and dissolve the Dibasic Sodium |
| | phosphate anhydrous, USP. |
| 8. | Mix Step 7 for NLT Ten (10) Minutes or until a clear |
| | solution is achieved. |
| 9. | To the Intermix Tank, add and dissolve the Sucralose, |
| | NF. |
| 10. | Mix Step 9 for NLT Ten (10) Minutes or until a clear |
| | solution is achieved. |
| 11. | Add 40% of total batch size of Purified water, USP, into |
| | the Main Process Tank. Start |
| | mixing with high shear mixer. |
| 12. | To the Main Process Tank, add the Simethicone Emulsion, |
| | USP slowly. |
| | Rinse the container using Purified water, USP: 1.00 kg. |
| | Add the rinse to Main Process Tank. |
| 13. | Mix Step 12 for NLT Ten (10) Minutes or until a uniform |
| | dispersion is achieved. |
| 14. | To the Main Process Tank, add the Chlorthalidone, USP |
| | slowly. |
| | Mix for NLT Thirty (30) Minutes or until a uniform |
| | dispersion is achieved. |
| | Suspension Description (White to off white |
| | suspension): |
| | Particle Size [D 90] of Chlorthalidone |
| | Dispersion: |
| 15. | Connect the outlet of the Main Process Tank to the inlet |
| | of the Admix Fast feed unit and outlet of the Admix Fast |
| | feed unit to the inlet of Main Process Tank. |
| | Start high shear mixing in the Main Process tank. Maintain |
| | the product temperature from 25-30° C. throughout the |
| | process. |
| | Start Admix Fast feed. |
| | Homogenize Chlorthalidone Dispersion until desired Particle |
| | size [D90: 40-90 um] is obtained. |
| | Suspension Description (White to off white |
| | suspension): |
| | Note: |
| | Do Not disconnect Admix Fast feed unit. |
| 16. | Transfer the dispersion from Step # 10 into Main Process |
| | Tank under stirring. Rinse the Intermix Tank with Purified |
| | Water, USP [5.00 kg] and add the rinse to the Main |
| | Process Tank under stirring. |
| 17. | Weigh the Glycerin Natural 99.7%, USP in Separate SS vessel. |
| 18. | To the SS vessel, add the Xanthan gum, NF slowly. |
| | Mix for NLT Ten (10) Minutes or until a uniform dispersion |
| | is achieved. |
| 19. | Transfer the dispersion from Step # 18 into Main Process |
| | Tank under stirring. |
| | Mix for NLT Sixty (60) Minutes until to get uniform suspension. |
| 20. | To the Main Process Tank, add the Tutti Frutti flavour. |
| | Rinse the SS vessel with Purified Water, USP [1.00 kg] and |
| | add the rinse to the Main Process Tank under stirring. |
| 21. | Add remaining quantity of Purified water to make up the volume |
| | to batch size. |
| | Mix for Ten (10) Minutes. |
| 22. | Pass the suspension through Fast feed Max (FFD-01) with |
| | Dynashear turned OFF for Thirty (30) minutes. |
| | pH (5.0-7.0): |
| | Suspension Description (White to off white |
| | suspension): |
| 23. | Close the Process Tank with a lid and label the tank with |
| | product name, Lot number, Quantity. |
| 24. | Withdraw approximately 400 grams of the following bulk pre- |
| | filtration samples from the Process Tank. |
| | Top, Middle and Bottom samples: Submit to QC for testing. |
| 25. | Hold the batch in the Process Tank for filling/packaging. |

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of“a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless context dictates or noted otherwise, all percentages are percent by weight.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A chlorthalidone suspension for oral administration, comprising:
water containing a dissolved buffer, a dissolved solubilizing and/or wetting agent, and at least one dissolved suspending agent;
wherein the buffer maintains a pH of the suspension between 5 and 7;
wherein the dissolved solubilizing and/or wetting agent, and/or the dissolved suspending agent are present in an amount to impart a viscosity of between 100-230 cps to the suspension; and
undissolved chlorthalidone particles having a particle size distribution d (0.9) of less than 90 μM, wherein the chlorthalidone is present in the suspension at a concentration of between 1 to 20 mg/mL and maintains, upon mixing to uniformity, a uniform dispersion in the suspension for at least 30 minutes.

2. The suspension of claim 1, wherein the buffer comprises a phosphate buffer, and/or wherein the pH of the suspension is between 5 and 6.

3. The suspension of claim 1, wherein the dissolved solubilizing and/or wetting agent is selected from the group consisting of glycerin, propylene glycol (PPG), poloxamer 188, poloxamer 124, and poloxamer 237.

4. The suspension of claim 1, wherein the suspending agent is a polysaccharide suspending agent and/or a synthetic suspending agent.

5. The suspension of claim 3, wherein the suspending agent is one or more selected from microcrystalline cellulose (MCC), acacia gum, tragacanth gum, a xanthan gum, starch, or alginates.

6. The suspension of claim 4, wherein the suspending agent is one or more selected from microcrystalline cellulose (MCC), acacia gum, tragacanth gum, a xanthan gum, starch, or alginates.

7. The suspension of claim 1, further comprising a sweetener, an anti-foam agent, and/or a flavoring agent.

8. The suspension of claim 1, wherein the viscosity of the suspension is between 130-190 cps.

9. The suspension of claim 1, wherein the chlorthalidone particles are milled chlorthalidone particles.

10. The suspension of claim 1, wherein the chlorthalidone particles have a particle size distribution d (0.9) of less than 40 μM.

11. The suspension of claim 1, wherein the chlorthalidone maintains, upon mixing to uniformity, a uniform dispersion in the suspension for at least 60 minutes.

12. The suspension of claim 1, wherein no less than 75% of all chlorthalidone particles dissolve within 60 minutes upon mixing with purified water.

13. The suspension of claim 1, wherein after storage at 40° C. and 25% or 25° C. and 40% relative humidity no less than 70% of all chlorthalidone particles dissolve within 60 minutes upon mixing with purified water.

14. The suspension of claim 1, wherein the suspension contains after storage at 40° C. and 25% relative humidity no more than 1.2 wt % total chlorthalidone degradation products of a total quantity of chlorthalidone in the suspension, and/or wherein the suspension contains after storage at 25° C. and 40% relative humidity no more than 0.9 wt % total chlorthalidone degradation products of a total quantity of chlorthalidone in the suspension.

15. A chlorthalidone suspension, comprising:
water containing a dissolved antimicrobial agent, a dissolved phosphate buffer, dissolved glycerin, and a dissolved xanthan gum;
wherein the glycerin and the xanthan gum impart a viscosity of between 100-230 cps to the suspension, and wherein the suspension has a pH of between 5 and 7;
undissolved milled or un-milled chlorthalidone particles having a particle size distribution d (0.9) of less than 90 μM, wherein the chlorthalidone is present in the suspension at a concentration of 5 mg/mL or 10 mg/mL; and
wherein the suspension maintains, upon mixing to uniformity, a uniform dispersion of the milled or un-milled chlorthalidone particles in the suspension for at least 30 minutes.

16. The suspension of claim 15, further comprising a sweetener, an anti-foaming agent, and/or a flavoring agent.

17. The suspension of claim 15, wherein the suspension contains after storage at 40° C. and 25% relative humidity no more than 1.2 wt % total chlorthalidone degradation products of a total quantity of chlorthalidone in the suspension, and/or wherein the suspension contains after storage at 25° C. and 40% relative humidity no more than 0.9 wt % total chlorthalidone degradation products of a total quantity of chlorthalidone in the suspension.

18. The suspension of claim 15, wherein after storage at 40° C. and 25% or 25° C. and 40% relative humidity no less than 70% of all chlorthalidone particles dissolve within 60 minutes upon mixing with purified water.

19. The suspension of claim 15, wherein the chlorthalidone particles are milled particles and have a particle size distribution d (0.9) of less than 40 μM.

20. The suspension of claim 15, wherein after storage at 40° C. and 25% or 25° C. and 40% relative humidity the viscosity of the suspension is between 130-190 cps.
